(19) United States
(12) Patent Application Publication
(10) Pub. No.: US 2016/0364525 A1
(43) Pub. Date: Dec. 15, 2016

(54) MEDICAL IMAGE PROCESSING APPARATUS AND MEDICAL IMAGE TRANSFER SYSTEM

(71) Applicant: Toshiba Medical Systems Corporation, Otawara-shi (JP)

(72) Inventors: Koji TAKEI, Nasushiobara (JP); Hirofumi ISHIHARA, Yaita (JP); Takumi KANEKO, Nasushiobara (JP); Hideaki KOBAYASHI, Otawara (JP); Takayuki KOJIMA, Sakura (JP); Yoshifumi YAMAGATA, Otawara (JP)

(73) Assignee: Toshiba Medical Systems Corporation, Otawara-shi (JP)

(21) Appl. No.: 15/175,585

(22) Filed: Jun. 7, 2016

(30) Foreign Application Priority Data
Jun. 9, 2015 (JP) 2015-116778
Jun. 3, 2016 (JP) 2016-111754

(51) Int. Cl. G06F 19/00; A61B 8/00; A61B 6/00; A61B 5/055; A61B 5/00; A61B 6/03

(52) U.S. Cl. CPC G06F 19/321; A61B 5/0013; A61B 6/032; A61B 6/56; A61B 5/055; A61B 8/56; A61B 6/4435; A61B 6/467; A61B 6/461; A61B 6/4476; A61B 6/5205

(57) ABSTRACT

According to one embodiment, a medical image processing apparatus includes memory circuitry, and transfer circuitry. The memory circuitry configured to store a plurality of medical images obtained by capturing a subject and a plurality of pieces of additional information respectively associated with the plurality of medical images. The transfer circuitry configured to transfer the plurality of medical images to an external apparatus in accordance with a predetermined transfer sequence. If the additional information associated with each medical image matches a predetermined determination condition, the transfer circuitry changes the predetermined transfer sequence, and transfers the plurality of medical images to the external apparatus.

### CROSS-REFERENCE TO RELATED APPLICATIONS

This application is based upon and claims the benefit of priority from Japanese Patent Application No. 2015-116778, filed Jun. 9, 2015; and No. 2016-111754, filed Jun. 3, 2016, the entire contents of all of which are incorporated herein by reference.

### FIELD

Embodiments described herein relate generally to a medical image processing apparatus and a medical image transfer system.

### BACKGROUND

Images generated by a medical image diagnosis apparatus (including a medical image processing apparatus) such as an X-ray computed tomography (CT) apparatus or magnetic resonance imaging (MRI) apparatus are transferred to an external apparatus such as a medical image analysis apparatus (for example, a workstation) or a medical image management system (for example, a picture archiving and communication system (PACS)), which is provided separately from the medical image diagnosis apparatus. For example, the images transferred to the medical image analysis apparatus are analyzed by a clinical application incorporated in the medical image analysis apparatus.

Based on, for example, the storage sequence of the images and the display sequence of an image list, the medical image diagnosis apparatus sets a transfer sequence of transferring the images to the medical image analysis apparatus. The medical image diagnosis apparatus transfers the image data to the medical image analysis apparatus in the set transfer sequence. Upon completion of transfer of all the images, the medical image analysis apparatus starts analysis by the clinical application.

However, the conventional medical image diagnosis apparatus can execute analysis by the clinical application only after completion of transfer of all the images. An increase in the number of images generated by the medical image diagnosis apparatus and the enlargement of the matrix size of each image are significant. Along with an increase in the number of images and the enlargement of the matrix size of each image, an image transfer time from the medical image diagnosis apparatus to the medical image analysis apparatus or medical image management system is prolonged. Consequently, it takes time for the clinical application to start analysis after the start of image transfer. That is, the problem that a long waiting time is needed before diagnosis arises. Furthermore, if it takes time to transfer images, the image transfer processing may be abandoned midway. This poses a problem that it is impossible to perform image analysis and diagnosis.

### BRIEF DESCRIPTION OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is a block diagram showing a medical image transfer system according to the first embodiment;

FIG. 2A is a table showing an example of an item (parameter) list which is stored in memory circuitry shown in FIG. 1 and is to be used to determine priority levels;

FIG. 2B is a table showing an example of an item (parameter) list which is stored in the memory circuitry shown in FIG. 1 and is to be used to determine priority levels;

FIG. 3 is a sequence chart showing a procedure until a medical image diagnosis apparatus executes a predetermined study, and transfers medical images across a plurality of series obtained in the study according to Example 1;

FIG. 4 is a schematic view showing the data structure of the medical images which are acquired by image acquisition processing in steps Sa1 to Sa3 and stored in the memory circuitry according to Example 1;

FIG. 5 is a flowchart illustrating a procedure of setting a transfer sequence according to Example 1;

FIG. 6 is a table showing an example of a transfer table in which transfer ordinal numbers generated by processing circuitry are respectively associated with volumes to be transferred according to Example 1;

FIG. 7 is a sequence chart showing a procedure until a medical image diagnosis apparatus executes a predetermined study, and transfers medical images across a plurality of series obtained in the study according to Example 2;

FIG. 8 is a schematic view showing the data structure of medical images which are acquired by image acquisition processing in steps Sb1 to Sb3 and stored in memory circuitry according to Example 2;

FIG. 9 is a flowchart illustrating a procedure of setting a transfer sequence according to Example 2;

FIG. 10 is a table showing an example of a transfer table in which transfer ordinal numbers generated by processing circuitry are respectively associated with frames to be transferred according to Example 2;

FIG. 11 is a sequence chart showing a procedure until a medical image diagnosis apparatus executes a predetermined study, and transfers medical images across a plurality of series obtained in the study according to Example 3;

FIG. 12 is a schematic view showing the data structure of medical images which are acquired by image acquisition processing in steps Sc1 to Sc3 and stored in memory circuitry according to Example 3;

FIG. 13 is a flowchart illustrating a procedure of setting a transfer sequence according to Example 3;

FIG. 14 is a table showing an example of a transfer table in which transfer ordinal numbers generated by processing circuitry are respectively associated with frames to be transferred according to Example 3;

FIG. 15 is a sequence chart showing a procedure until a medical image diagnosis apparatus executes a predetermined study, and transfers medical images across a plurality of series obtained in the study according to Example 4;

FIG. 16 is a schematic view showing the data structure of medical images which are acquired by image acquisition processing in steps Sd1 to Sd3 and stored in memory circuitry according to Example 4;

FIG. 17 is a flowchart illustrating a procedure of setting a transfer sequence according to Example 4;

FIG. 18 is a table showing an example of a transfer table in which transfer ordinal numbers generated by processing circuitry are respectively associated with frames to be transferred according to Example 4;

FIG. 19 is a sequence chart showing a procedure until a medical image diagnosis apparatus executes a predetermined study, and transfers a plurality of medical images belonging to the same series obtained in the study according to Example 5;

FIG. 20 is a schematic view showing the data structure of the medical images which are acquired by image acquisition processing in steps Se1 to Se3 and stored in memory circuitry according to Example 5;

FIG. 21 is a flowchart illustrating a procedure of setting a transfer sequence according to Example 5;

FIG. 22 is a table showing an example of a transfer table in which transfer sequence ordinal numbers generated by processing circuitry are respectively associated with frames to be transferred according to Example 5;

FIG. 23 is a view showing an example of a display mode of dual energy images transferred from the medical image diagnosis apparatus to a workstation according to Example 5;

FIG. 24 is a sequence chart showing a procedure until a medical image diagnosis apparatus executes predetermined studies, and transfers medical images across the plurality of studies obtained in the studies according to Example 6;

FIG. 25 is a schematic view showing the data structure of medical images which are acquired by image acquisition processing in steps Sf1 to Sf3 and stored in memory circuitry according to Example 6;

FIG. 26 is a flowchart illustrating a procedure of setting a transfer sequence according to Example 6;

FIG. 27 is a table showing an example of a transfer table in which transfer ordinal numbers generated by processing circuitry are respectively associated with frames to be transferred according to Example 6;

FIG. 28 is a view showing an example of a display mode of images before/after surgery transferred from the medical image diagnosis apparatus to a workstation according to Example 6;

FIG. 29 is a sequence chart showing a procedure until a medical image diagnosis apparatus executes a predetermined study, and transfers medical images across a plurality of series obtained in the study according to Example 7;

FIG. 30 is a block diagram showing a medical image transfer system according to the second embodiment; and

FIG. 31 is a sequence chart showing a procedure until a medical image diagnosis apparatus executes a predetermined study, and transfers medical images across a plurality of series obtained in the study according to the second embodiment.

### DETAILED DESCRIPTION

In general, according to one embodiment, a medical image processing apparatus includes memory circuitry, and transfer circuitry. The memory circuitry configured to store a plurality of medical images obtained by capturing a subject and a plurality of pieces of additional information respectively associated with the plurality of medical images. The transfer circuitry configured to transfer the plurality of medical images to an external apparatus in accordance with a predetermined transfer sequence. If the additional information associated with each medical image matches a predetermined determination condition, the transfer circuitry changes the predetermined transfer sequence, and transfers the plurality of medical images to the external apparatus.

A medical image processing apparatus and a medical image transfer system including the medical image processing apparatus according to embodiments will be described below with reference to the accompanying drawings. Note that in the following description, the same reference numerals denote components having almost the same functions and arrangements, and a repetitive description thereof will be made, only as needed.

### First Embodiment

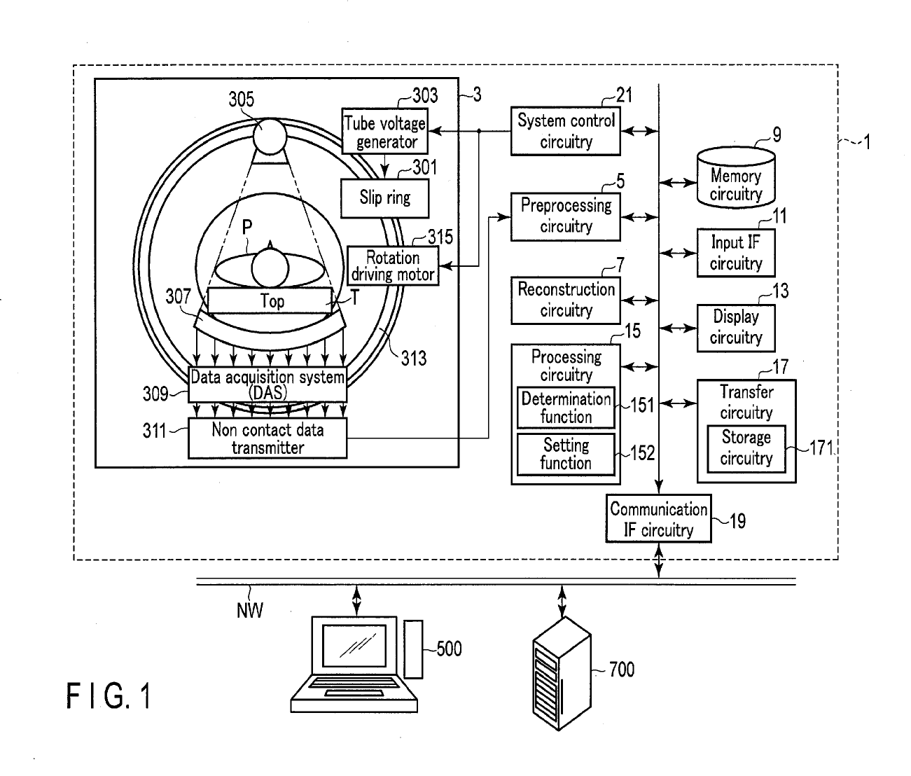

FIG. 1 is a block diagram showing a medical image transfer system according to the first embodiment.

As shown in FIG. 1, the medical image transfer system includes a medical image diagnosis apparatus (including a medical image processing apparatus) 1, a workstation (WS) 500 for executing image analysis, and a PACS 700 for saving medical images. For example, the medical image diagnosis apparatus 1, WS 500, and PACS 700 are interconnected via a network NW. Note that FIG. 1 shows the arrangement of an X-ray CT apparatus as an example of the medical image diagnosis apparatus 1. Note also that the first embodiment shows the arrangement of the X-ray CT apparatus as an example of the medical image diagnosis apparatus 1 but the present invention is not limited to this. For example, the medical image diagnosis apparatus 1 may be an MRI apparatus, X-ray diagnosis apparatus, nuclear medicine diagnosis apparatus, or ultrasonic diagnosis apparatus. The same applies to a subsequent embodiment.

As shown in FIG. 1, the medical image diagnosis apparatus 1 includes a gantry 3, preprocessing circuitry 5, reconstruction circuitry 7, memory circuitry 9, input interface (IF) circuitry 11, display circuitry 13, processing circuitry 15, transfer circuitry 17, communication IF circuitry 19, and system control circuitry 21.

The gantry 3 includes a slip ring 301, a tube voltage generator 303, an X-ray tube 305, an X-ray detector 307, a data acquisition system (DAS) 309, and a non-contact data transmitter 311. The gantry 3 also includes a rotating ring 313, a ring support mechanism that supports the rotating ring 313 to be rotatable about the body axis (Z-axis) of a subject, and a rotation driving motor (electric motor) 315 that drives the rotation of the rotating ring 313. A top T on which a subject P can be placed is inserted to the opening portion of the rotating ring 313. The top T is supported by a bed (not shown) to be movable along the central axis of the rotating ring 313. At this time, the top T is positioned so that the body axis of the subject P placed on the top T coincides with the central axis of the rotating ring 313. The rotating ring 313 incorporates the tube voltage generator 303, the X-ray tube 305, the DAS 309, the non-contact data transmitter 311, a cooling device (not shown), a gantry control device (not shown), and the like. The gantry control device is formed from, for example, a processor, a memory, and the like.

Under the control of the system control circuitry 21, the tube voltage generator 303 generates a tube voltage to be applied to the X-ray tube 305 and a filament current to be supplied to the X-ray tube 305. For example, the tube voltage generator 303 periodically changes the tube voltage to be supplied to the X-ray tube 305 between a high voltage (for example, 140 kV) and a low voltage (for example, 80 kV).

The X-ray tube 305 receives application of the tube voltage and supply of the filament current from the tube voltage generator 303 via the slip ring 301. The X-ray tube 305 emits X-rays from the X-ray focus to the subject P placed on the top T. The X-ray tube 305 generates X-rays having an energy spectrum corresponding to the tube voltage applied by the tube voltage generator 303. For example, the X-ray tube 305 generates X-rays having an energy spectrum corresponding to each of the high voltage and low voltage applied by the tube voltage generator 303. An X-ray radiation range is indicated by two-dot dashed line shown in FIG. 1.

The X-ray detector 307 is attached to the rotating ring 313 at a position and angle so as to face the X-ray tube 305 through a rotation axis. The X-ray detector 307 has a plurality of light-receiving bands. In this case, assume that one light-receiving band forms one channel. A plurality of channels are arrayed two-dimensionally in the two directions, i.e., the Z-direction (slice direction) and an arc direction (channel direction) indicated by an arc that is perpendicular to the rotation axis, is centered on the focus of the emitted X-rays, and has, as its radius, the distance from that center to the center of the light-receiving band for one channel. The DAS 309 is connected to the output side of the X-ray detector 307. The X-ray detector 307 arrays the plurality of light-receiving bands in line. At this time, the plurality of light-receiving bands are arrayed one-dimensionally in almost the arc direction along the channel direction. The plurality of light-receiving bands may be arrayed two-dimensionally in the two directions, i.e., the channel direction and the slice direction. That is, the two-dimensional array is formed by arraying, in the slice direction, a plurality of arrays each including a plurality of channels arrayed one-dimensionally along the channel direction. The X-ray detector 307 including the two-dimensional light-receiving band array may be formed by arraying, in the slice direction, a plurality of arrays each including the plurality of light-receiving bands arrayed one-dimensionally in almost the arc direction.

The DAS 309 is attached, for each channel, with an I-V converter that converts a current signal from each channel of the X-ray detector 307 into a voltage, an integrator that periodically integrates these voltage signals in synchronism with an X-ray irradiation period, an amplifier that amplifies an output signal from the integrator, and an analog-digital converter that converts an output signal from the amplifier into a digital signal. The DAS 309 transmits output data (raw data) to the preprocessing circuitry 5 via the non-contact data transmitter 311 using magnetic transmission/reception or optical transmission/reception.

The preprocessing circuitry 5 performs preprocessing for the raw data output from the non-contact data transmitter 311. The preprocessing includes, for example, logarithmic conversion processing for the raw data, sensitivity nonuniformity correction processing between channels, and processing of correcting an extreme decrease in signal intensity or signal dropout caused by an X-ray strong absorber, mainly a metal portion. The preprocessing circuitry 5 transmits, to the reconstruction circuitry 7 and the memory circuitry 9, data (projection data) having undergone the preprocessing immediately before reconstruction processing in association with data representing view angles at the time of data acquisition.

Note that the projection data indicates a set of data values each corresponding to the intensity of X-rays having passed through the subject. For the sake of descriptive convenience, a set of projection data throughout a plurality of channels which are almost simultaneously acquired by one shot at the same view angle will be referred to as a projection data set hereinafter. View angles are obtained by representing, by angles in the range of 0° to 360°, the respective positions on a circular orbit obtained when the X-ray tube 305 revolves about the rotation axis, with the angle of the uppermost portion on the circular orbit in an upward vertical direction from the rotation axis being 0°. Note that projection data of a projection data set which corresponds to each channel is identified by a view angle, cone angle, and channel number.

The reconstruction circuitry 7 is used to reconstruct a nearly cylindrical volume data by the Feldkamp method or the cone beam reconstruction method based on the projection data set acquired at view angles in the range of 360° or (180°+fan angle) and transmitted from the preprocessing circuitry 5, and is implemented by, for example, a memory and a predetermined processor. In addition, the reconstruction circuitry 7 is used to reconstruct a two-dimensional CT image (tomographic image to be simply referred to as a CT image hereinafter) from the above projection data set by, for example, the fan beam reconstruction method (also called the fan beam convolution back projection method), filtered back projection (FBP), or a successive approximation reconstruction method, and is implemented by, for example, a memory and a predetermined processor. The Feldkamp method is a reconstruction method to be used when projection rays intersect a reconstruction plane like a cone beam. The Feldkamp method is an approximate image reconstruction method of performing convolution by regarding a projection beam as a fan projection beam on the premise that the cone angle is small, and performing back projection in a scan along a ray. The cone beam reconstruction method is a reconstruction method which corrects projection data in accordance with the angle of a ray relative to a reconstruction plane as a method of suppressing cone angle errors more than the Feldkamp method. The reconstruction circuitry 7 transmits the reconstructed volume data to the memory circuitry 9. The reconstruction circuitry 7 transmits the reconstructed CT image to the memory circuitry 9 and the processing circuitry 15.

The reconstruction circuitry 7 reconstructs a CT image complying with the Digital Imaging Communication in Medicine (DICOM) standard. That is, the CT image contains additional information associated with the CT image in addition to image information. The additional information includes at least one of data indicating the characteristics and attributes (a data format, a series ID, a comment added to a series, a frame (medical image) number, and the like) of the corresponding image, information (a tube voltage, a tube current, contrast absence/presence, a scan name, reconstruction conditions (scan conditions), and the like) about imaging, examination information (a study ID, an examination date/time, an examination portion, an examination apparatus name, a person taking charge of an examination, a phase (cardiac phase), order conditions, and the like) about an examination (diagnosis) using the image, patient information (a patient ID, a patient name, the date of birth, sex, and weight of the patient, patient coordinates obtained by setting a predetermined position of the patient as an origin, and the like).

Note that the reconstruction circuitry 7 includes dual-energy image reconstruction circuitry that reconstructs two-dimensional distribution tomographic images of X-ray tube voltage-dependent information associated with the distribution of atoms, that is, tomographic images of so-called dual-energy imaging.

The memory circuitry 9 includes a solid state drive (SSD) and a hard disk drive (HDD) that can store a relatively large amount of data. The memory circuitry 9 stores a CT image reconstructed by the reconstruction circuitry 7 and additional information associated with the CT image. The memory circuitry 9 stores in advance information (determination conditions) necessary to determine transfer priority levels (transfer ordinal numbers) from the additional information associated with the CT image. The determination conditions indicate items (parameters) to be used to determine the priority levels. The medical image diagnosis apparatus 1 according to the first embodiment collates the determination conditions with additional information associated with each of a plurality of medical images, and sets a transfer ordinal number for each of the plurality of medical images.

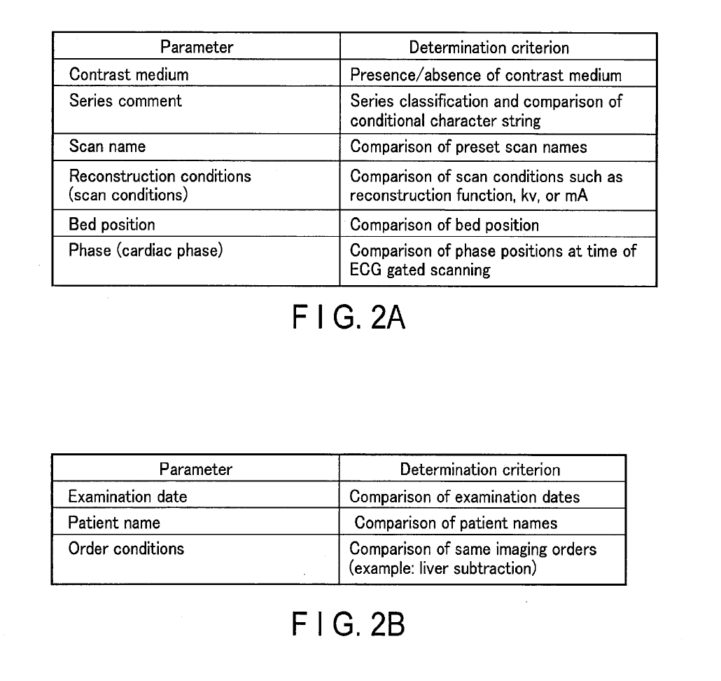

FIGS. 2A and 2B are tables each showing an example of an item (parameter) list to be used to determine priority levels, which is stored in the memory circuitry 9. For example, as shown in FIG. 2A, the memory circuitry 9 stores, as the items to be used to determine the priority levels, contrast absence/presence, a comment (series comment) added to a series, a scan name, reconstruction conditions (scan conditions), a bed position, a phase (cardiac phase), and the like. The items shown in FIG. 2A are used to, for example, determine image transfer priority levels in the same examination (series unit). Alternatively, as shown in FIG. 2B, the memory circuitry 9 stores an examination date, a patient name, order conditions, and the like as items to be used to determine the priority levels. The items shown in FIG. 2B are used to, for example, determine image transfer priority levels in follow-up (study unit).

The memory circuitry 9 stores the projection data transmitted from the preprocessing circuitry 5 and the volume data reconstructed by the reconstruction circuitry 7. The memory circuitry 9 stores a control program for controlling the timing of applying each of the high voltage and the low voltage to the X-ray tube 305.

Note that the memory circuitry 9 may use an optical disk such as a magnetooptical disk, compact disc (CD), or digital versatile disc (DVD) instead of the magnetic disk such as the HDD. The saving area of the memory circuitry 9 may be included in the medical image diagnosis apparatus 1 or an external storage device connected by the network NW.

The input IF circuitry 11 serves as an interface for inputting a command or the like corresponding to a user operation. For example, by inputting a command or the like corresponding to a user operation via the input IF circuitry 11, information (determination conditions) necessary for the user to determine the transfer priority levels (transfer ordinal numbers) is input. The input IF circuitry 11 includes, for example, a keyboard, a mouse, a touch panel, a trackball, and various buttons.

The display circuitry 13 displays, for example, the CT image, the three-dimensional image, and the like on a display device. As the display device, a cathode ray tube display (CRT), liquid crystal display (LCD), organic electro luminescence display (OELD), or plasma display can be used, as needed.

The processing circuitry 15 includes, as hardware components, a predetermined processor such as a central processing unit (CPU) or micro processing unit (MPU), and predetermined memories such as a read-only memory (ROM) and random access memory (RAM). The memory of the processing circuitry 15 stores a determination program. The processing circuitry 15 reads out the determination program stored in the predetermined memory, and executes it, thereby implementing a determination function 151. By implementing the determination function 151, the processing circuitry 15 determines whether medical images each satisfying the determination conditions stored in the memory circuitry 9 or input via the input IF circuitry 11 exist in the memory circuitry 9.

The memory of the processing circuitry 15 stores a setting program. The processing circuitry 15 reads out the setting program stored in the predetermined memory, and executes it, thereby implementing a setting function 152. By implementing the setting function 152, if it is determined that medical images each satisfying the determination conditions exist in the memory circuitry 9, the processing circuitry 15 collates additional information associated with each medical image with the determination conditions, and sets a transfer sequence of a plurality of medical image data associated with pieces of additional information each matching the determination conditions. For example, the processing circuitry 15 determines a transfer priority level for each of the plurality of medical image data which are associated with the pieces of additional information each matching the determination conditions, and sets the transfer ordinal number of each of the plurality of medical image data in accordance with the priority level. The processing circuitry 15 determines a transfer priority level for each of the plurality of medical image data across a plurality of series, and sets a transfer ordinal number of each of the plurality of medical image data based on the priority levels. For example, the processing circuitry 15 determines a transfer priority level for each volume, and sets a transfer ordinal number for each volume. The processing circuitry 15 determines a transfer priority level for each frame, and sets a transfer ordinal number for each frame.

More specifically, the processing circuitry 15 sets a transfer sequence so as to alternately transfer a plurality of frame images between different medical images of different series among the plurality of medical images respectively associated with the pieces of matching additional information. Alternatively, the processing circuitry 15 sets a transfer sequence so as to alternately transfer a plurality of frame images for each frame image between difference medical images of the same series among the plurality of medical images respectively associated with the pieces of matching additional information.

If it is determined that the medical images each satisfying the determination conditions exist in the memory circuitry 9, the transfer circuitry 17 transfers the plurality of medical images to at least one of the WS 500 and PACS 700 in the transfer sequence set by the processing circuitry 15. The transfer circuitry 17 transfers the plurality of medical images via the communication IF circuitry 19. If it is determined that medical images each of which does not satisfy the determination conditions exist in the memory circuitry 9, the transfer circuitry 17 transfers the medical images by a normal transfer method.

More specifically, the transfer circuitry 17 includes storage circuitry 171 for storing transfer requests about the plurality of medical images to an external apparatus. If each of pieces of additional information respectively associated with medical images corresponding to transfer requests stored in the storage circuitry 171 matches the predetermined determination conditions, the transfer circuitry 17 transfers the plurality of medical images by changing a transfer sequence corresponding to the reception sequence of the transfer requests stored in the storage circuitry 171. If each of the pieces of additional information respectively associated with the images corresponding to the transfer requests stored in the storage circuitry 171 does not match the predetermined determined conditions, the transfer circuitry 17 transfers the plurality of medical images in a transfer sequence corresponding to the reception sequence of the transfer requests stored in the storage circuitry 171 as a predetermined transfer sequence.

The medical image diagnosis apparatus sets a transfer sequence of transferring the medical images to the medical image analysis apparatus based on the storage sequence of the medical images, the display sequence of the medical image list, and the like. The medical image diagnosis apparatus transfers the medical images to the medical image analysis apparatus in accordance with the set transfer sequence. Upon completion of transfer of all the images, the medical image analysis apparatus starts analysis by the clinical application.

The communication IF circuitry 19 communicates with an external apparatus by wired or wireless connection. The external apparatus is, for example, another modality, a server included in a system such as a radiological information system (RIS), hospital information system (HIS), or PACS, or another workstation. In the first embodiment, the communication IF circuitry 19 communicates with the WS 500 and the PACS 700.

The system control circuitry 21 includes, as hardware components, a predetermined processor such as a CPU or MPU and predetermined memories such as a ROM and RAM. The memory of the system control circuitry 21 stores a control program. The system control circuitry 21 reads out the control program stored in the predetermined memory, and executes it, thereby controlling operations and processes between the plurality of units of the internal arrangement of the gantry 3 and the preprocessing circuitry 5, reconstruction circuitry 7, memory circuitry 9, input IF circuitry 11, display circuitry 13, processing circuitry 15, transfer circuitry 17, and communication IF circuitry 19. For example, the system control circuitry 21 controls power supply from the slip ring 301 to the tube voltage generator 303 so as to perform imaging according to a predetermined scan sequence. More specifically, the system control circuitry 21 controls the tube voltage generator 303 so as to periodically change the tube voltage of the X-ray tube 305 between the high voltage (for example, 140 kV) and the low voltage (for example, 80 kV). Note that the high and low voltages may be referred to as high and low energy levels, respectively.

The system control circuitry 21 controls the rotation driving unit 315 to rotate the rotating ring 313 at a speed as high as 0.4 sec/rotation or the like. The system control circuitry 21 controls a top driving unit (not shown) to move the top T. The movement of the top T moves the subject P placed on the top T along the rotation axis.

Transfer of medical images by the medical image diagnosis apparatus 1 according to the first embodiment will be described using practical examples.

### Example 1

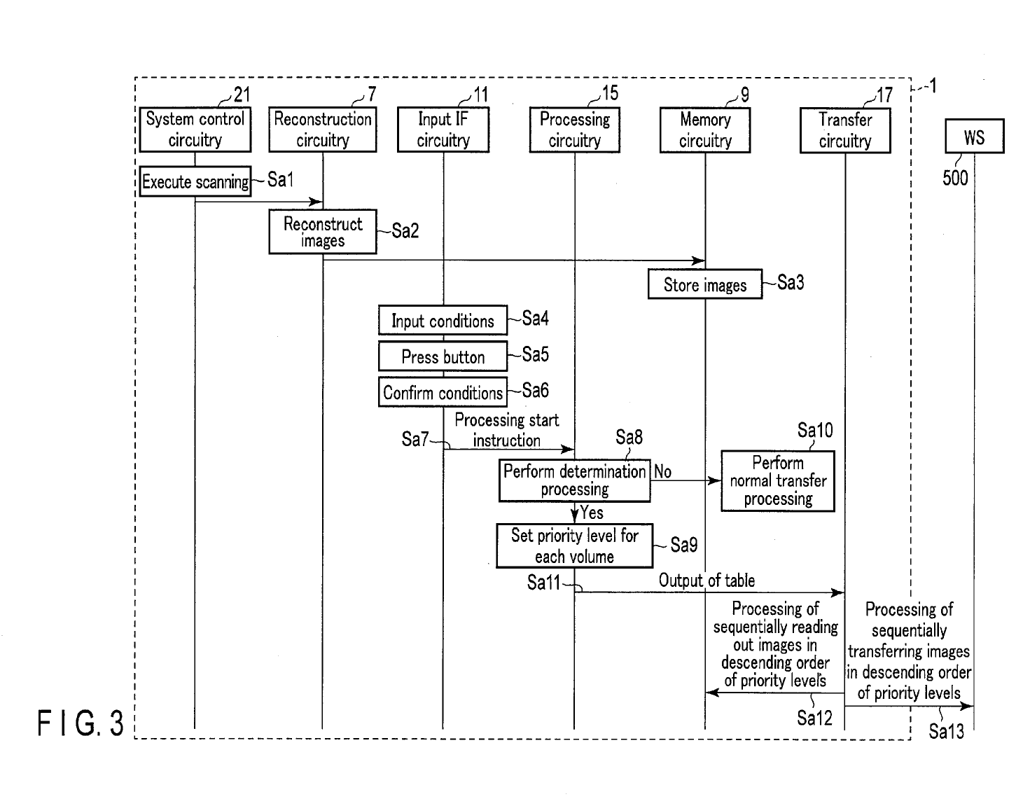

FIG. 3 is a sequence chart showing a procedure until a medical image diagnosis apparatus 1 executes a predetermined study (for example, an electrocardiogram (ECG) gated cardiac examination using a contrast medium), and transfers data across a plurality of series obtained in the study according to Example 1. Transfer processing executed by the medical image diagnosis apparatus 1 will be described below with reference to FIG. 3. Example 1 will describe, as an example, a case in which the medical image diagnosis apparatus 1 transfers images to a WS 500. The same applies to subsequent examples and embodiment. The medical image diagnosis apparatus 1 according to Example 1 can set a transfer ordinal number for each volume across the plurality of series. As an example, a description will be given by assuming priority transfer (volume unit) for subtraction processing using a non-contrast image and a contrast image.

As shown in FIG. 3, in step Sa1, after patient conditions and predetermined imaging conditions (a diagnosis portion, an imaging method, a tube voltage, a tube current, reconstruction parameters, contrast absence/presence, an imaging range (bed position), a cardiac phase, and the like) are input, system control circuitry 21 executes ECG gated pre-contrast imaging and ECG gated post-contrast imaging within a predetermined range (for example, a bed position of 0 to 300 mm) in accordance with a predetermined imaging sequence. As a result, for example, volumes 1, 2, and 3 (as raw data) belonging to series 1 are acquired in pre-contrast imaging, and volumes 4, 5, and 6 (as raw data) belonging to series 2 are acquired in post-contrast imaging.

In step Sa2, the reconstruction circuitry 7 executes image reconstruction processing using the respective acquired volumes and the set reconstruction parameters, and generates volumes 1, 2, and 3 belonging to series 1 by non-contrast imaging, and volumes 4, 5, and 6 belonging to series 2 by contrast imaging. In step Sa3, each generated volume of each series is added with additional information including “volume identification number, contrast absence/presence, bed position (imaging range), and cardiac phase in ECG waveform”, and stored in the memory circuitry 9, as needed.

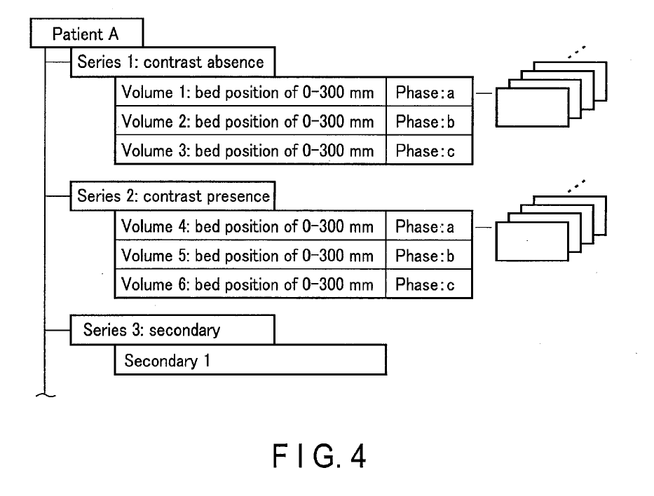

FIG. 4 is a schematic view showing the data structure of the image data acquired by the image acquisition processing in steps Sa1 to Sa3 and stored in the memory circuitry 9. In the example shown in FIG. 4, a plurality of volumes acquired at the same bed position (imaging range) and at a plurality of phases (cardiac phases) are stored in series 1. A plurality of volumes acquired at the same bed position (imaging range) and at a plurality of phases (cardiac phases) are stored in series 2. As a result, in series 1 and 2, the plurality of frame images are stored with respect to the same slice (multi-frame format).

Referring to FIG. 4, volumes 1, 2, . . . are labeled. For example, volume 1 belonging to series 1 (contrast absence, that is, before administration of a contrast medium) is added with additional information including “contrast absence, bed position of 0-300 mm, and phase a”. Volume 2 belonging to series 1 is added with additional information including “contrast absence, bed position of 0-300 mm, and phase b”. Volume 3 belonging to series 1 is added with additional information including “contrast absence, bed position of 0-300 mm, and phase c”. Volume 4 belonging to series 2 (contrast presence, that is, after administration of a contrast medium) is added with additional information including “contrast presence, bed position of 0-300 mm, and phase a”. Volume 5 belonging to series 2 is added with additional information including “contrast presence, bed position of 0-300 mm, and phase b”. Volume 6 belonging to series 2 is added with additional information including “contrast presence, bed position of 0-300 mm, and phase c”. Note that series 3 includes, for example, a secondary image, that is, a screen capture image.

As shown in FIG. 3, in step Sa4, the user inputs determination conditions necessary to set transfer priority levels (transfer ordinal numbers) via input IF circuitry 11. For example, the user inputs, as determination conditions, “contrast examination, same bed position (same imaging range), and same phase” via the input IF circuitry 11. Alternatively, the user may select preset conditions as “priority transfer (volume unit) for subtraction processing”. In step Sa5, the user presses a processing start button via the input IF circuitry 11. In step Sa6, using pressing of the processing start button as a trigger, the input determination conditions are confirmed. In step Sa1, using pressing of the processing start button as a trigger, the input IF circuitry 11 outputs a processing start instruction to processing circuitry 15.

After pressing of the processing start button, the processing circuitry 15 determines in step Sa8 whether medical images each satisfying the determination conditions exist in memory circuitry 9. If the processing circuitry 15 determines that medical images each satisfying the determination conditions exist in the memory circuitry 9 (YES in step Sa8), it collates, in step Sa9, the confirmed determination conditions with the additional information associated with each of the plurality of volumes. Based on the collation result, the processing circuitry 15 sets a transfer priority level for each of the plurality of volumes belonging to each series acquired in the study.

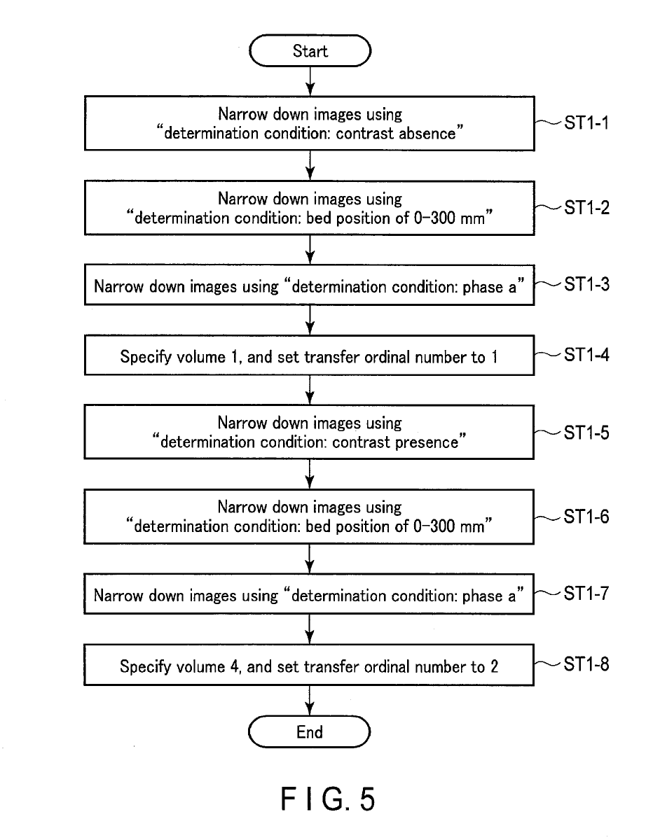

That is, as shown in FIG. 5, if the conditions “contrast examination, same bed position (same imaging range), and same phase” are confirmed as determination conditions, the processing circuitry 15 narrows down whether there are volumes corresponding to pieces of additional information each satisfying the determination conditions. More specifically, in step ST1-1, medical images are narrowed down to those associated with “additional information: before administration of contrast medium” using “determination condition: contrast absence”. Furthermore, in step ST1-2, the medical images are narrowed down to those associated with “additional information: bed position of 0-300 mm” using “determination condition: bed position of 0-300 mm”. Furthermore, in step ST1-3, the medical images are narrowed down to those associated with “additional information: phase a” using “determination condition: phase a”. With these operations, in step ST1-4, volume 1 belonging to series 1 is specified as a transfer target satisfying the determination conditions, and the transfer ordinal number of volume 1 is set to 1.

In step ST1-5, the medical images are narrowed down to those associated with “additional information: after administration of contrast medium” using “determination condition: contrast presence”. Furthermore, in step ST1-6, the medical images are narrowed down to those associated with “additional information: bed position of 0-300 mm” using “determination condition: bed position of 0-300 mm”. Furthermore, in step ST1-7, the medical images are narrowed down to those associated with “additional information: phase a” using “determination condition: phase a”. With these operations, in step ST1-8, volume 4 belonging to series 2 is specified as a transfer target satisfying the determination conditions, and the transfer ordinal number of volume 4 is set to 2.

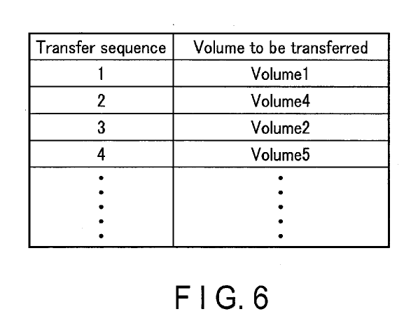

The same processing as that shown in FIG. 5 is repeatedly executed to set a transfer ordinal number of 3 for volume 2 having the additional information satisfying the conditions “contrast absence, bed position of 0-300 mm, and cardiac phase b” in the pre-contrast series, and set a transfer ordinal number of 4 for volume 5 having the additional information satisfying the conditions “contrast presence, bed position of 0-300 mm, and cardiac phase b” in the post-contrast series. Furthermore, a transfer ordinal number of 5 is set for volume 3 having the additional information satisfying the conditions “contrast absence, bed position of 0-300 mm, and cardiac phase c” in the pre-contrast series, and a transfer ordinal number of 6 is set for volume 6 having the additional information satisfying the conditions “contrast presence, bed position of 0-300 mm, and cardiac phase c” in the post-contrast series. This generates a transfer table, shown in FIG. 6, in which the transfer ordinal numbers are respectively associated with the volumes to be transferred.

The transfer sequence shown in FIG. 6 is an example. The present invention is not limited to this. For example, the transfer sequence may be set to “volume 4→volume 1 . . . .”. If the medical images each added with “additional information: phase b” are preferentially transferred, the transfer sequence may be set to “volume 2→volume 5 . . . ” or “volume 5→volume 2 . . . .” If the medical images each added with “additional information: phase c” are preferentially transferred, the transfer sequence may be set to “volume 3→volume 6 . . . ” or “volume 6→volume 3 . . . .”

In step Sa11, the processing circuitry 15 outputs the transfer table to transfer circuitry 17. In step Sa12, the transfer circuitry 17 sequentially reads out the volumes from the memory circuitry 9 in descending order of transfer priority levels in accordance with the received transfer table. The memory circuitry 9 outputs the plurality of corresponding volumes to the transfer circuitry 17 in response to the image readout processing from the transfer circuitry 17. In step Sa13, for example, the transfer circuitry 17 transfers, to the WS 500, the volumes read out in accordance with the transfer table.

If the processing circuitry 15 determines in step Sa8 that medical images each of which does not satisfy the determination conditions exist in the memory circuitry 9 (NO in step Sa8), it transfers, in step Sa10, by a normal transfer method (normal transfer), the medical images each of which does not satisfy the determination conditions. More specifically, if it is determined that medical images each of which does not satisfy the determination conditions exist in the memory circuitry 9, the processing circuitry 15 outputs a transfer instruction to the transfer circuitry 17 to transfer the medical images. Using reception of the transfer instruction as a trigger, the transfer circuitry 17 reads out the medical images from the memory circuitry 9 based on a predetermined condition, for example, in ascending order of the storage time in the memory circuitry 9. In response to the image readout processing from the transfer circuitry 17, the memory circuitry 9 outputs, to the transfer circuitry 17, the medical images each of which does not satisfy the determination conditions. For example, the transfer circuitry 17 transfers the readout medical images to the WS 500.

Note that the transfer circuitry 17 may transfer, in an arbitrary sequence input by an operator or the like, the medical images each of which does not satisfy the determination conditions. Medical images to be transferred by the normal transfer method may be read out and transferred for each volume, or read out and transferred for each frame included in the volume.

Furthermore, if medical images each of which does not satisfy the determination conditions exist in the memory circuitry 9 after transferring the medical images having higher priority levels (after step Sa13), the remaining medical images may be transferred by the normal transfer method. The same applies to the subsequent examples and embodiment.

With the above-described arrangement, the following effects can be obtained.

The medical image diagnosis apparatus 1 according to Example 1 includes the memory circuitry 9, processing circuitry 15, and transfer circuitry 17. The memory circuitry 9 stores volumes 1, 2, and 3 belonging to series 1 and obtained by non-contrast imaging, volumes 4, 5, and 6 belonging to series 2 and obtained by contrast imaging, and a plurality of pieces of additional information respectively associated with the volumes. The processing circuitry 15 collates each of the pieces of additional information with the predetermined determination conditions input via the input IF circuitry 11, determines a transfer priority level for each volume, and sets a transfer ordinal number for each volume based on the determined priority level. For example, the processing circuitry 15 sets a transfer sequence so as to preferentially transfer volumes for which the WS 500 executes subtraction processing (that is, so as to preferentially transfer pre- and post-contrast volumes at the same bed position and the same cardiac phase across the different series). The transfer circuitry 17 transfers the plurality of volumes to the WS 500 in the transfer sequence. This makes it possible to preferentially transfer volumes necessary for subtraction processing. As a result, the medical image diagnosis apparatus 1 according to Example 1 can shorten the time from image transfer to diagnosis, as compared with the conventional technique.

Note that the system control circuitry 21 uses the bed position as additional information for performing imaging according to the predetermined imaging sequence and setting the priority levels. The present invention, however, is not limited to this. The system control circuitry 21 may perform imaging and setting operations using patient coordinates obtained by setting a predetermined position of the patient as an origin.

### Example 2

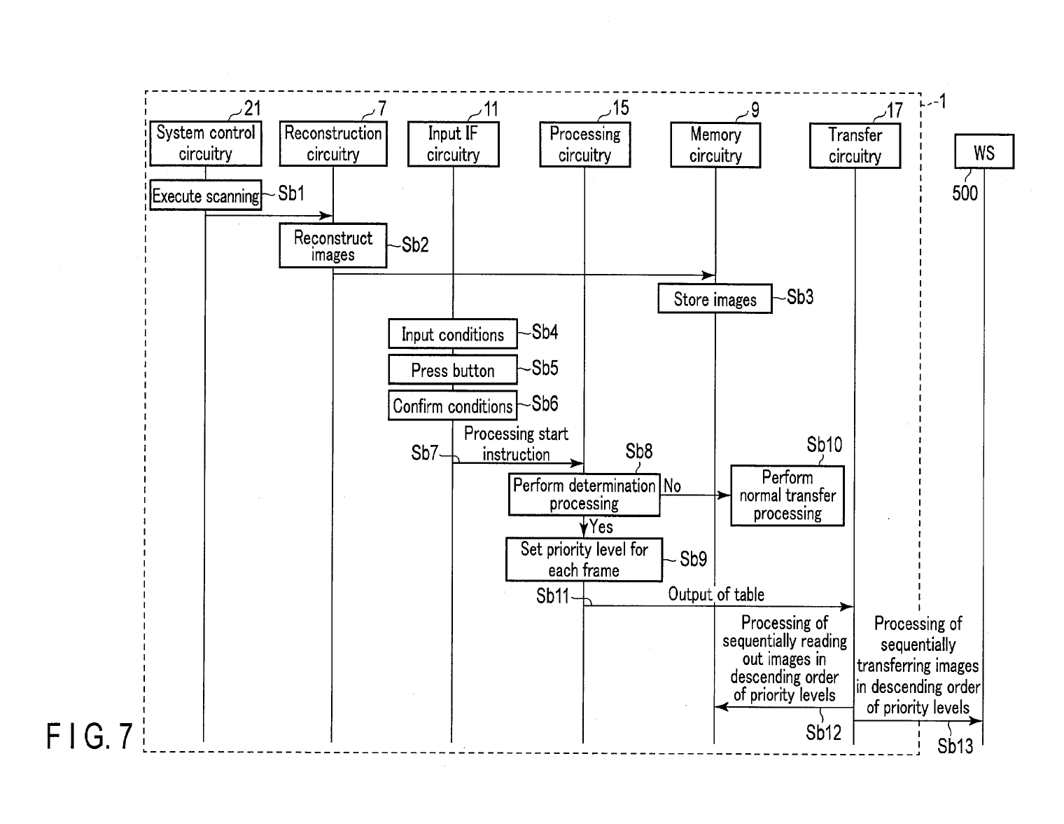

FIG. 7 is a sequence chart showing a procedure until a medical image diagnosis apparatus 1 executes a predetermined study (for example, an examination of a circulatory system using a contrast medium), and transfers data across a plurality of series obtained in the study according to Example 2. Transfer processing executed by the medical image diagnosis apparatus 1 will be described below with reference to FIG. 7. The medical image diagnosis apparatus 1 according to Example 2 can set a transfer ordinal number for each frame included in volumes across the plurality of series. As an example, a description will be given by assuming priority transfer (frame unit) for subtraction processing. Note that points common to the above example will not be described in detail, and will be described, as needed.

As shown in FIG. 7, in step Sb1, after patient conditions and predetermined imaging conditions (a diagnosis portion, an imaging method, a tube voltage, a tube current, reconstruction parameters, contrast absence/presence, an imaging range (bed position), a cardiac phase, and the like) are input, system control circuitry 21 executes imaging within a predetermined range (for example, a bed position of 0 to 300 mm) in accordance with a predetermined imaging sequence. As a result, for example, volume 1 (as raw data) belonging to series 1 is acquired in pre-contrast imaging, and volumes 2, 3, and 4 (as raw data) belonging to series 2 are acquired in post-contrast imaging. Note that each volume is formed from a plurality of two-dimensional data in frame units.

In step Sb2, reconstruction circuitry 7 executes image reconstruction processing using the respective acquired volumes and the set reconstruction parameters, and generates volume 1 belonging to series 1 by non-contrast imaging and volumes 2, 3, and 4 belonging to series 2 by contrast imaging. In step Sb3, each generated volume of each series is added with additional information including “volume identification number, frame identification number, contrast absence/presence, and bed position (imaging range)”, and stored in memory circuitry 9, as needed.

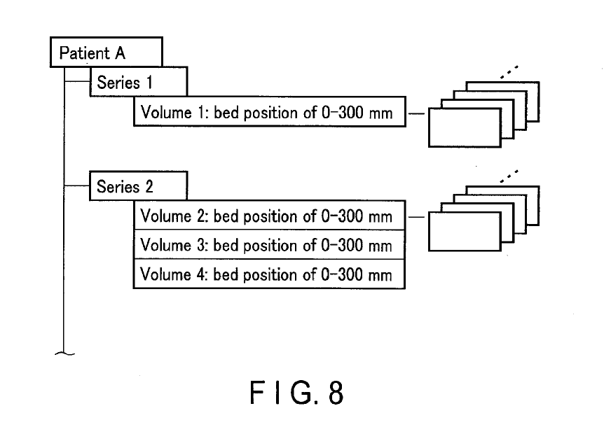

FIG. 8 is a schematic view showing the data structure of image data acquired by the image acquisition processing in steps Sb1 to Sb3 and stored in the memory circuitry 9. In the example shown in FIG. 8, one volume is stored in series 1. As a result, in series 1, one frame image is stored with respect to one slice (single-frame format). A plurality of volumes acquired at the same bed position (imaging range) are stored in series 2. As a result, in series 2, a plurality of frame images are stored with respect to the same slice (multi-frame format).

Referring to FIG. 8, volumes 1, 2, . . . are labeled. For example, volume 1 belonging to series 1 (contrast absence) is added with additional information including “contrast absence and bed position of 0-300 mm”. Each of volumes 2, 3, and 4 belonging to series 2 (contrast presence) is added with additional information including “contrast presence and bed position of 0-300 mm”.

As shown in FIG. 7, in step Sb4, the user inputs determination conditions necessary to set transfer priority levels (transfer ordinal numbers) via input IF circuitry 11. For example, the user inputs, as determination conditions, “contrast examination, same bed position (same imaging range), and same slice” via the input IF circuitry 11. Alternatively, the user may select preset conditions as “priority transfer (frame unit) for subtraction processing”. In step Sb5, the user presses a processing start button via the input IF circuitry 11. In step Sb6, using pressing of the processing start button as a trigger, the input determination conditions are confirmed. In step Sb7, using pressing of the processing start button as a trigger, the input IF circuitry 11 outputs a processing start instruction to processing circuitry 15.

After pressing of the processing start button, the processing circuitry 15 collates, in step Sb8, the confirmed determination conditions with the additional information associated with each of the plurality of volumes. Based on the collation result, the processing circuitry 15 sets a transfer priority level for each of the plurality of frames belonging to each series acquired in the study.

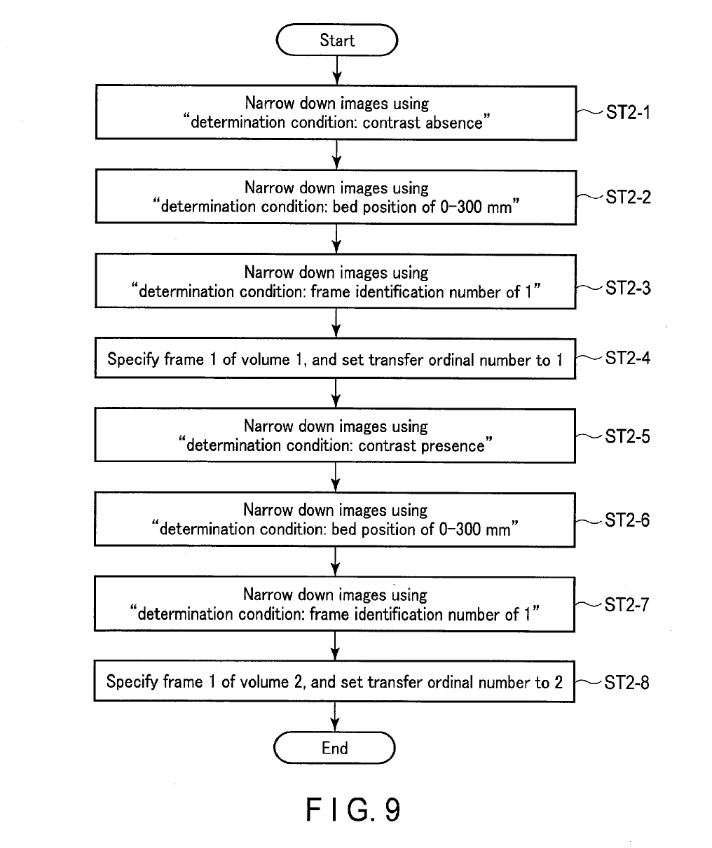

That is, as shown in FIG. 9, if the conditions “contrast examination, same bed position (same imaging range), and same slice” are confirmed as determination conditions, the processing circuitry 15 narrows down whether there are volumes corresponding to pieces of additional information each satisfying the determination conditions. More specifically, in step ST2-1, medical images are narrowed down to those associated with “additional information: before administration of contrast medium” using “determination condition: contrast absence”. Furthermore, in step ST2-2, the medical images are narrowed down to those associated with “additional information: bed position of 0-300 mm” using “determination condition: bed position of 0-300 mm”. Furthermore, in step ST2-3, the medical images are narrowed down to those associated with “additional information: frame 1” using “determination condition: frame identification number of 1”. With these operations, in step ST2-4, frame 1 of volume 1 belonging to series 1 is specified as a transfer target satisfying the determination conditions, and the transfer ordinal number of frame 1 of volume 1 is set to 1.

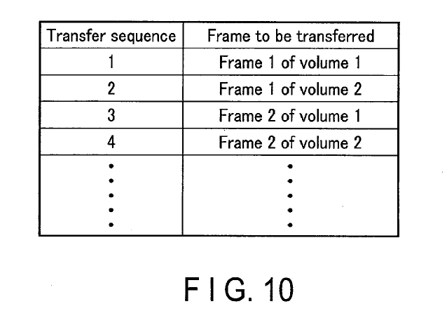

In step ST2-5, the medical images are narrowed down to those associated with “additional information: after administration of contrast medium” using “determination condition: contrast presence”. Furthermore, in step ST2-6, the medical images are narrowed down to those associated with “additional information: bed position of 0-300 mm” using “determination condition: bed position of 0-300 mm”. Furthermore, in step ST2-7, the medical images are narrowed down to those associated with “additional information: frame 1” using “determination condition: frame identification number of 1”. With these operations, in step ST2-8, frame 1 of volume 2 belonging to series 2 is specified as a transfer target satisfying the determination conditions, and the transfer ordinal number of frame 1 of volume 2 is set to 2. The same processing as that shown in FIG. 9 is repeatedly executed. That is, a transfer sequence is set so as to preferentially transfer the medical image data of the same slice at the same bed position, for which subtraction processing is executable. This generates a transfer table, shown in FIG. 10, in which transfer ordinal numbers are respectively associated with frames to be transferred.

The transfer sequence shown in FIG. 10 is an example. The present invention is not limited to this. For example, the transfer sequence may be changed, as needed, to “frame 1 of volume 2→frame 1 of volume 1 . . . ”, “frame 2 of volume 1→frame 2 of volume 2 . . . ”, or the like.

With the above-described arrangement, the following effects can be obtained.

The medical image diagnosis apparatus 1 according to Example 2 includes the memory circuitry 9, the processing circuitry 15, and transfer circuitry 17. The memory circuitry 9 stores volume 1 belonging to series 1 and obtained by non-contrast imaging, volumes 2, 3, and 4 belonging to series 2 and obtained by contrast imaging, and a plurality of pieces of additional information respectively associated with the volumes. The processing circuitry 15 collates each of the plurality of pieces of additional information with the predetermined determination conditions input via the input IF circuitry 11, and determines a transfer priority level for each of the plurality of frames included in each volume, thereby setting a transfer ordinal number for each of the plurality of frames based on the determined priority level. For example, the processing circuitry 15 sets a transfer sequence so as to preferentially transfer frames for which a WS 500 executes subtraction processing (that is, so as to preferentially transfer pre- and post-contrast frames of the same slice at the same bed position across the different series). The transfer circuitry 17 transfers the plurality of frames to the WS 500 in the transfer sequence. This makes it possible to preferentially transfer frames necessary for subtraction processing. As a result, since the medical image diagnosis apparatus 1 according to Example 2 performs transfer processing for each frame, it can shorten the time until subtraction processing is executed, as compared with a case in which transfer processing is performed for each volume. Even if image transfer is interrupted midway, processing (display) can be executed using medical images received so far.

Note that the system control circuitry 21 uses the bed position as additional information for performing imaging according to the predetermined imaging sequence and setting the priority levels. The present invention, however, is not limited to this. The system control circuitry 21 may perform imaging and setting operations using, for example, patient coordinates obtained by setting a predetermined position of the patient as an origin.

### Example 3

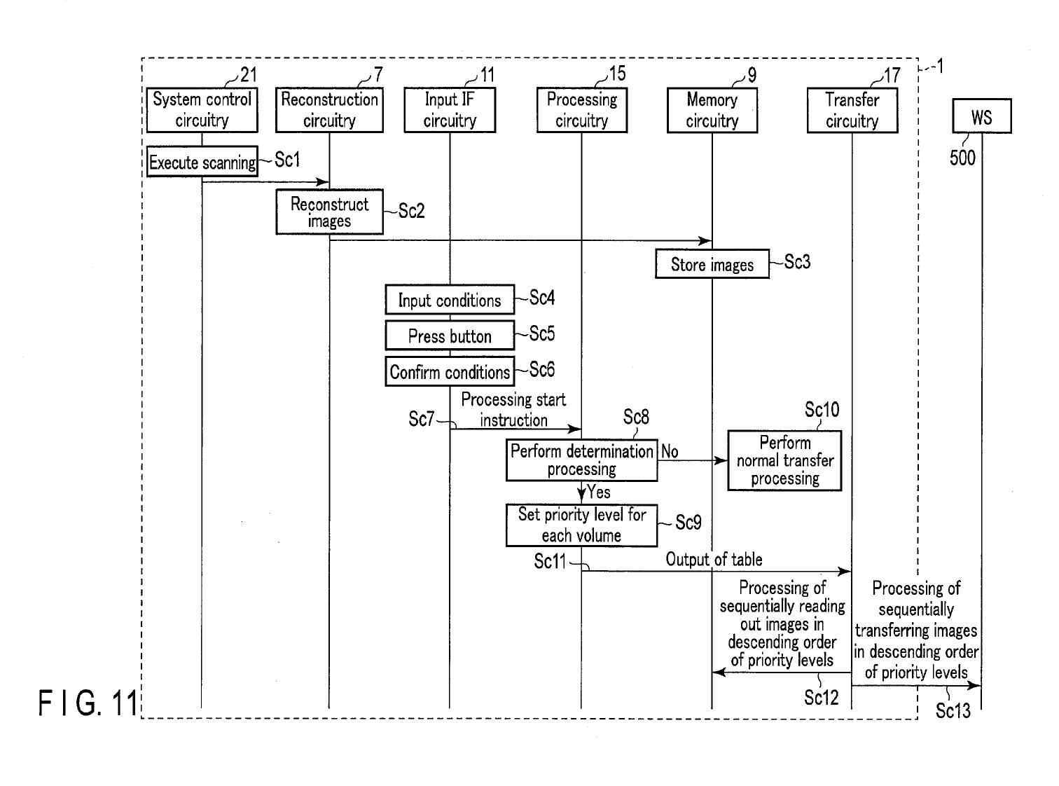

FIG. 11 is a sequence chart showing a procedure until a medical image diagnosis apparatus 1 executes a predetermined study (for example, an examination of a circulatory system using a contrast medium), and transfers data across a plurality of series obtained in the study according to Example 3. Transfer processing executed by the medical image diagnosis apparatus 1 will be described below with reference to FIG. 11. The medical image diagnosis apparatus 1 according to Example 3 can set a transfer ordinal number for each volume across the plurality of series. As an example, a description will be given by assuming priority transfer (volume unit) for subtraction processing. Note that points common to the above examples will not be described in detail, and will be described, as needed.

As shown in FIG. 11, in step Sc1, after patient conditions and predetermined imaging conditions (a diagnosis portion, an imaging method, a tube voltage, a tube current, reconstruction parameters, contrast absence/presence, an imaging range (bed position), a cardiac phase, and the like) are input, system control circuitry 21 executes imaging within predetermined ranges (for example, bed positions of 0 to 300 mm, 300 to 600 mm, and 600 to 900 mm) in accordance with a predetermined imaging sequence. As a result, for example, volumes 1, 2, and 3 (as raw data) belonging to series 1 are acquired in pre-contrast imaging, and volumes 4, 5, 6, 7, and 8 (as raw data) belonging to series 2 are acquired in post-contrast imaging.

In step Sc2, reconstruction circuitry 7 executes image reconstruction processing using the respective acquired volumes and the set reconstruction parameters, and generates volumes 1, 2, and 3 belonging to series 1 by non-contrast imaging, and volumes 4, 5, 6, 7, and 8 belonging to series 2 by contrast imaging. In step Sc3, each generated volume of each series is added with additional information including “volume identification number, contrast absence/presence, and bed position (imaging range)”, and stored in memory circuitry 9, as needed.

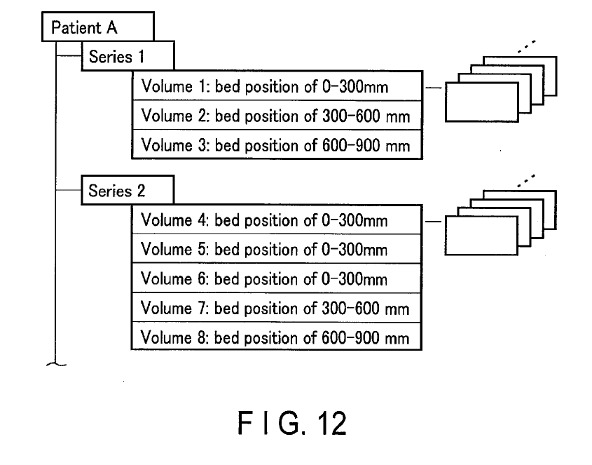

FIG. 12 is a schematic view showing the data structure of the image data acquired by the image acquisition processing in steps Sc1 to Sc3 and stored in the memory circuitry 9. In the example shown in FIG. 12, three volumes of different bed positions are stored in series 1. A plurality of volumes acquired at the same bed position (imaging range) and two volumes of different bed positions are stored in series 2.

Referring to FIG. 12, volumes 1, 2, . . . are labeled. For example, volume 1 belonging to series 1 (contrast absence) is added with additional information including “contrast absence and bed position of 0-300 mm”. Volume 2 belonging to series 1 is added with additional information including “contrast absence and bed position of 300-600 mm”. Volume 3 belonging to series 1 is added with additional information including “contrast absence and bed position of 600-900 mm”. Each of volumes 4, 5, and 6 belonging to series 2 (contrast presence) is added with additional information including “contrast presence and bed position of 0-300 mm”. Volume 7 belonging to series 2 is added with additional information including “contrast presence and bed position of 300-600 mm”. Volume 8 belonging to series 2 is added with additional information including “contrast presence and bed position of 600-900 mm”.

As shown in FIG. 11, in step Sc4, the user inputs determination conditions necessary to set transfer priority levels (transfer ordinal numbers) via input IF circuitry 11. For example, the user inputs, as determination conditions, “contrast examination and same bed position (same imaging range)” via the input IF circuitry 11. Alternatively, the user may select preset conditions as “priority transfer (volume unit) for subtraction processing”. In step Sc5, the user presses a processing start button via the input IF circuitry 11. In step Sc6, using pressing of the processing start button as a trigger, the input determination conditions are confirmed. In step Sc7, using pressing of the processing start button as a trigger, the input IF circuitry 11 outputs a processing start instruction to processing circuitry 15.

After pressing of the processing start button, the processing circuitry 15 collates, in step Sc8, the confirmed determination conditions with additional information associated with each of the plurality of volumes. Based on the collation result, the processing circuitry 15 sets a transfer priority level for each of the plurality of volumes belonging to each series acquired in the study.

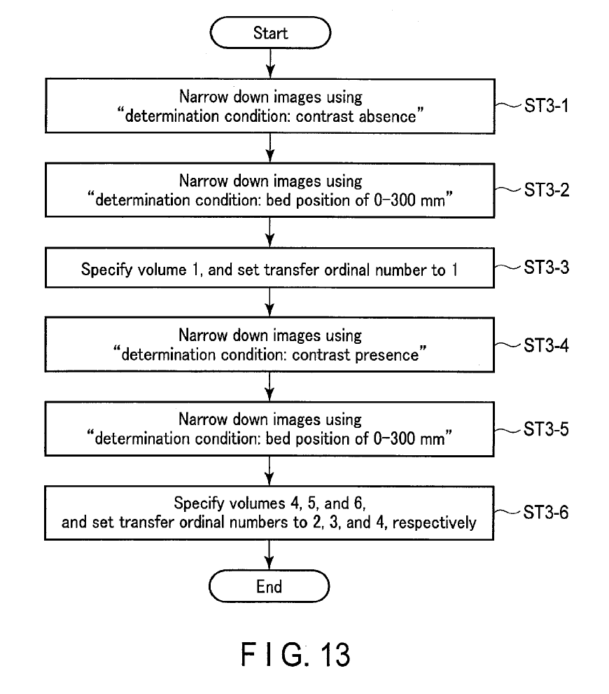

That is, as shown in FIG. 13, if the conditions “contrast examination and same bed position (same imaging range)” are confirmed as determination conditions, the processing circuitry 15 narrows down whether there are volumes corresponding to pieces of additional information each satisfying the determination conditions. More specifically, in step ST3-1, medical images are narrowed down to those associated with “additional information: before administration of contrast medium” using “determination condition: contrast absence”. Furthermore, in step ST3-2, the medical images are narrowed down to those associated with “additional information: bed position of 0-300 mm” using “determination condition: bed position of 0-300 mm”. With these operations, in step ST3-3, volume 1 belonging to series 1 is specified as a transfer target satisfying the determination conditions, and the transfer ordinal number of volume 1 is set to 1.

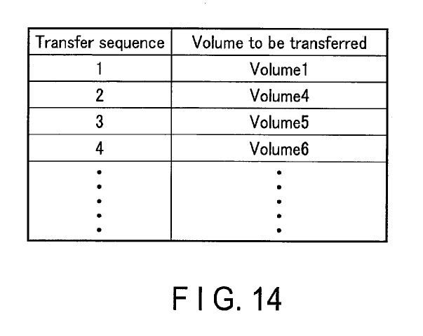

In step ST3-4, the medical images are narrowed down to those associated with “additional information: after administration of contrast medium” using “determination condition: contrast presence”. Furthermore, in step ST3-5, the medical images are narrowed down to those associated with “additional information: bed position of 0-300 mm” using “determination condition: bed position of 0-300 mm”. With these operations, in step ST3-6, volumes 4, 5, and 6 belonging to series 2 are specified as transfer targets each satisfying the determination conditions, and the transfer ordinal number of volume 4 is set to 2. The transfer ordinal number of volume 5 is set to 3. The transfer ordinal number of volume 6 is set to 4. This generates a transfer table, shown in FIG. 14, in which the transfer ordinal numbers are respectively associated with the volumes to be transferred.

The transfer sequence shown in FIG. 14 is an example. The present invention is not limited to this. For example, the transfer sequence may be changed, as needed, to “volume 4→volume 1 . . . ” or “volume 5→volume 1 . . . .”

With the above-described arrangement, the following effects can be obtained.

The medical image diagnosis apparatus 1 according to Example 3 includes the memory circuitry 9, the processing circuitry 15, and transfer circuitry 17. The memory circuitry 9 stores volumes 1, 2, and 3 belonging to series 1 and obtained by non-contrast imaging, volumes 4, 5, 6, 7, and 8 belonging to series 2 and obtained by contrast imaging, and a plurality of pieces of additional information respectively associated with the volumes. The processing circuitry 15 collates each of the pieces of additional information with predetermined determination conditions input via the input IF circuitry 11, specifies volumes each satisfying the predetermined conditions, and sets a transfer priority level for each of the specified volumes. For example, the processing circuitry 15 sets a transfer sequence so as to preferentially transfer volumes for which a WS 500 executes subtraction processing (that is, so as to preferentially transfer pre- and post-contrast volumes at the same bed position across the different series). The transfer circuitry 17 transfers the plurality of volumes to the WS 500 in the transfer sequence. This makes it possible to preferentially transfer volumes necessary for subtraction processing. As a result, even if the medical image diagnosis apparatus 1 according to Example 3 has a plurality of volumes of different bed positions (imaging ranges) in the same series, it can shorten the time from image transfer to diagnosis, as compared with the conventional technique.

Note that the system control circuitry 21 uses the bed position as additional information for performing imaging according to the predetermined imaging sequence and setting the priority levels. The present invention, however, is not limited to this. The system control circuitry 21 may perform imaging and setting operations using patient coordinates obtained by setting a predetermined position of the patient as an origin.

### Example 4

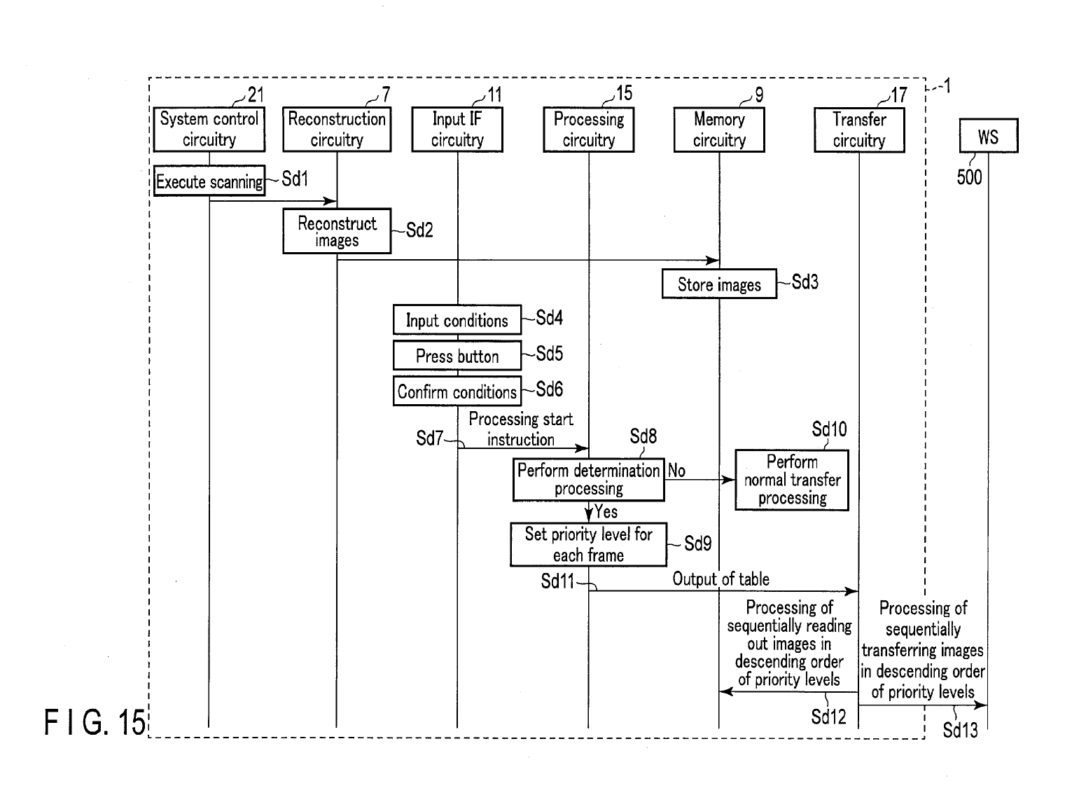

FIG. 15 is a sequence chart showing a procedure until a medical image diagnosis apparatus 1 executes a predetermined study (for example, an examination of a circulatory system using a contrast medium), and transfers data across a plurality of series obtained in the study according to Example 4. Transfer processing executed by the medical image diagnosis apparatus 1 will be described below with reference to FIG. 15. The medical image diagnosis apparatus 1 according to Example 4 can set a transfer ordinal number for each frame across a plurality of series. As an example, a description will be given by assuming priority transfer (frame unit) for subtraction processing. Note that points common to the above examples will not be described in detail, and will be described, as needed.

As shown in FIG. 15, in step Sd1, after patient conditions and predetermined imaging conditions (a diagnosis portion, an imaging method, a tube voltage, a tube current, reconstruction parameters, contrast absence/presence, an imaging range (bed position), a cardiac phase, and the like) are input, system control circuitry 21 executes imaging within predetermined ranges (for example, a bed position of −10, 0, 10, 20, and 30 mm) in accordance with a predetermined imaging sequence. As a result, for example, frames 1, 2, and 3 (as raw data) belonging to series 1 are acquired in pre-contrast imaging, and frames 4, 5, 6, 7, and 8 (as raw data) belonging to series 2 are acquired in post-contrast imaging.

In step Sd2, reconstruction circuitry 7 executes image reconstruction processing using the respective acquired volumes and the set reconstruction parameters, and generates frames 1, 2, and 3 belonging to series 1 by non-contrast imaging, and frames 4, 5, 6, 7, and 8 belonging to series 2 by contrast imaging. In step Sd3, each generated frame of each series is added with additional information including “frame identification number, contrast absence/presence, and bed position (imaging range)”, and stored in memory circuitry 9, as needed.

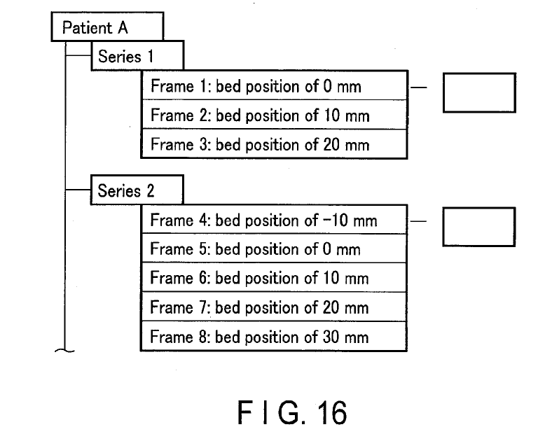

FIG. 16 is a schematic view showing the data structure of the image data acquired by the image acquisition processing in steps Sd1 to Sd3 and stored in the memory circuitry 9. In the example shown in FIG. 16, a plurality of frames of different bed positions are stored in series 1. Furthermore, a plurality of frames of different bed positions are stored in series 2, similarly to series 1.

Referring to FIG. 16, frames 1, 2, . . . are labeled. For example, frame 1 belonging to series 1 (contrast absence) is added with additional information including “contrast absence and bed position of 0 mm”. Frame 2 belonging to series 1 is added with additional information including “contrast absence and bed position of 10 mm”. Frame 3 belonging to series 1 is added with additional information including “contrast absence and bed position of 20 mm”. Frame 4 belonging to series 2 (contrast presence) is added with additional information including “contrast presence and bed position of −10 mm”. Frame 5 belonging to series 2 is added with additional information including “contrast presence and bed position of 0 mm”. Frame 6 belonging to series 2 is added with additional information including “contrast presence and bed position of 10 mm”. Frame 7 belonging to series 2 is added with additional information including “contrast presence and bed position of 20 mm”. Frame 8 belonging to series 2 is added with additional information including “contrast presence and bed position of 30 mm”.

As shown in FIG. 15, in step Sd4, the user inputs determination conditions necessary to set transfer priority levels (transfer ordinal numbers) via input IF circuitry 11. For example, the user inputs, as determination conditions, “contrast examination, same bed position, and same slice” via the input IF circuitry 11. Alternatively, the user may select preset conditions as “priority transfer (frame unit) for subtraction processing”. In step Sb5, the user presses a processing start button via the input IF circuitry 11. In step Sd6, using pressing of the processing start button as a trigger, the input determination conditions are confirmed. In step Sd7, using pressing of the processing start button as a trigger, the input IF circuitry 11 outputs a processing start instruction to processing circuitry 15.

After pressing of the processing start button, the processing circuitry 15 collates, in step Sd8, the confirmed determination conditions with the additional information associated with each of the plurality of frames. Based on the collation result, the processing circuitry 15 sets a transfer priority level for each of the plurality of frames belonging to each series acquired in the study.

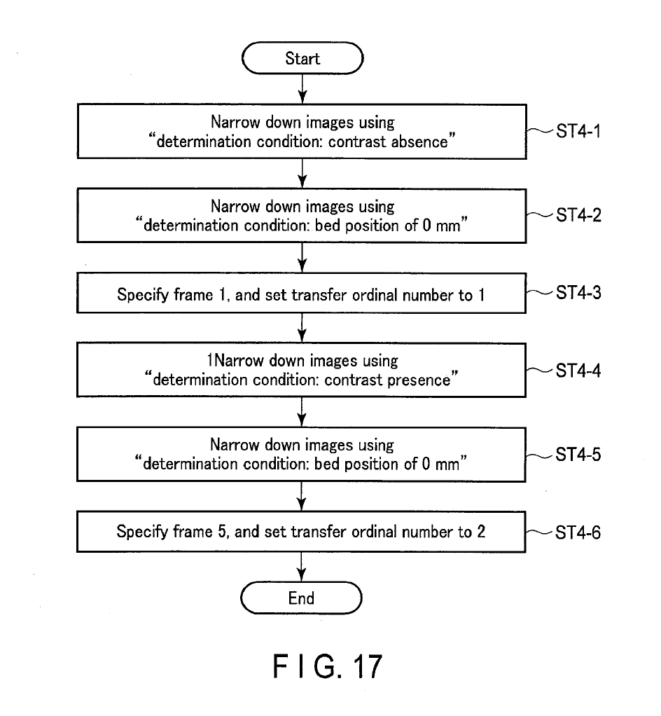

That is, as shown in FIG. 17, if the conditions “contrast examination, same bed position, and same slice” are confirmed as determination conditions, the processing circuitry 15 narrows down whether there are frames corresponding to pieces of additional information each satisfying the determination conditions. More specifically, in step ST4-1, medical images are narrowed down to those associated with “additional information: before administration of contrast medium” using “determination condition: contrast absence”. Furthermore, in step ST4-2, the medical images are narrowed down to those associated with “additional information: bed position of 0 mm” using “determination condition: bed position of 0 mm”. With these operations, in step ST4-3, frame 1 belonging to series 1 satisfying the determination conditions is specified, and the transfer ordinal number of frame 1 is set to 1.

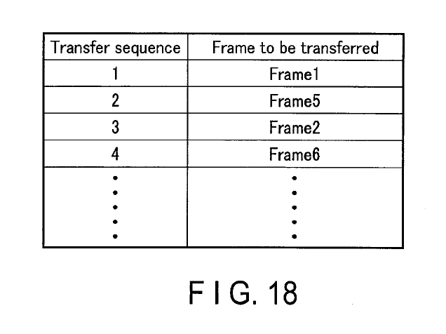

Furthermore, in step ST4-4, medical images are narrowed down to those associated with “additional information: after administration of contrast medium” using “determination condition: contrast presence”. Furthermore, in step ST4-5, the medical images are narrowed down to those associated with “additional information: bed position of 0 mm” using “determination condition: bed position of 0 mm”. With these operations, in step ST4-6, frame 5 belonging to series 2 satisfying the determination conditions is specified, and the transfer ordinal number of frame 5 is set to 2. The same processing as that shown in FIG. 17 is repeatedly executed to set a transfer ordinal number of 3 for frame 2 having the additional information satisfying the conditions “contrast absence and bed position of 10 mm” in the pre-contrast series, and set a transfer ordinal number of 4 for frame 6 having the additional information satisfying the conditions “contrast presence and bed position of 10 mm” in the post-contrast series. Furthermore, a transfer ordinal number of 5 is set for frame 3 having the additional information satisfying the conditions “contrast absence and bed position of 20 mm” in the pre-contrast series, and a transfer ordinal number of 6 is set for frame 7 having the additional information satisfying the conditions “contrast presence and bed position of 20 mm” in the post-contrast series. This generates a transfer table, shown in FIG. 18, in which the transfer ordinal numbers are respectively associated with the frames to be transferred.

The transfer sequence shown in FIG. 18 is an example. The present invention is not limited to this. For example, the transfer sequence may be changed, as needed, to “frame 2→frame 6 . . . ”, “frame 3→frame 7 . . . ”, or the like.

With the above-described arrangement, the following effects can be obtained.

The medical image diagnosis apparatus 1 according to Example 4 includes the memory circuitry 9, the processing circuitry 15, and transfer circuitry 17. The memory circuitry 9 stores frames 1, 2, and 3 belonging to series 1 and obtained by non-contrast imaging, frames 4, 5, 6, 7, and 8 belonging to series 2 and obtained by contrast imaging, and a plurality of pieces of additional information respectively associated with the frames. The processing circuitry 15 collates each of the plurality of pieces of additional information with predetermined conditions input via the input IF circuitry 11, and determines a transfer priority level for each frame, thereby setting a transfer ordinal number for each frame based on the determined priority level. For example, the processing circuitry 15 sets a transfer sequence so as to preferentially transfer frames for which a WS 500 executes subtraction processing (that is, so as to preferentially transfer pre- and post-contrast frames of the same slice at the same bed position across the different series). The transfer circuitry 17 transfers the plurality of frames to the WS 500 in the transfer sequence. This makes it possible to preferentially transfer frames necessary for subtraction processing. As a result, the medical image diagnosis apparatus 1 according to Example 4 implements proper transfer in accordance with image data. Furthermore, the medical image diagnosis apparatus 1 performs transfer processing for each frame, it can shorten the time until subtraction processing is executed. Even if image transfer is interrupted midway, subtraction processing can be executed using medical images received so far.

Note that the system control circuitry 21 uses the bed position as additional information for performing imaging according to the predetermined imaging sequence and setting the priority levels. The present invention, however, is not limited to this. The system control circuitry 21 may perform imaging and setting operations using, for example, patient coordinates obtained by setting a predetermined position of the patient as an origin.

### Example 5

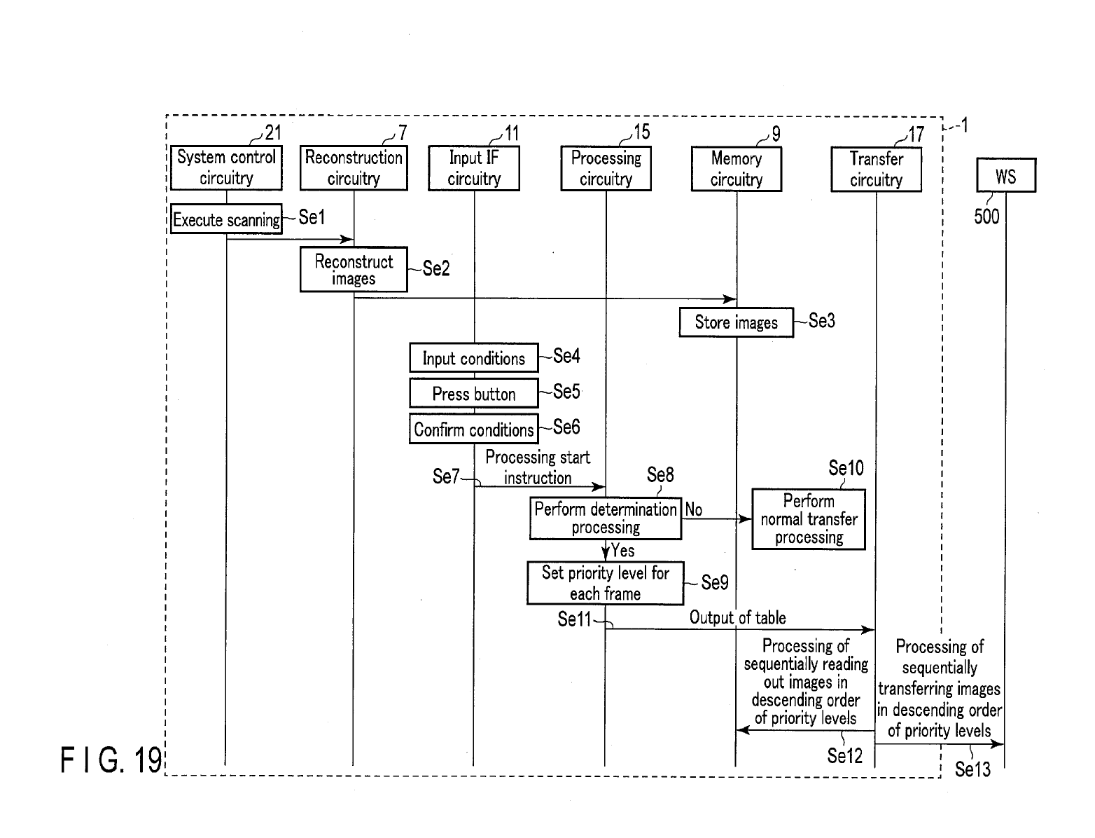

FIG. 19 is a sequence chart showing a procedure until a medical image diagnosis apparatus 1 executes a predetermined study (for example, an examination of outputting a plurality of volumes in the same examination, such as dual energy imaging), and transfers a plurality of data belonging to the same series obtained in the study according to Example 5. Transfer processing executed by the medical image diagnosis apparatus 1 will be described below with reference to FIG. 19. The medical image diagnosis apparatus 1 according to Example 5 can set a transfer ordinal number for each frame included in volumes in the same series. As an example, a description will be given by assuming priority transfer (frame unit) for analysis processing of dual energy images. Note that points common to the above examples will not be described in detail, and will be described, as needed.

As shown in FIG. 19, in step Se1, after patient conditions and predetermined imaging conditions (a diagnosis portion, an imaging method, a tube voltage, a tube current, reconstruction parameters, contrast absence/presence, an imaging range (bed position), a cardiac phase, and the like) are input, system control circuitry 21 executes imaging within a predetermined range (for example, a bed position of 0 to 160 mm) in accordance with a predetermined imaging sequence. As a result, for example, volumes 1 and 2 (as raw data) belonging to series 1 are acquired. Note that each volume is formed from a plurality of two-dimensional data in frame units.

In step Set, reconstruction circuitry 7 executes image reconstruction processing using the respective acquired volumes and the set reconstruction parameters, and generates volumes 1 and 2 belonging to series 1. In step Se3, each generated volume of each series is added with additional information including “volume identification number, frame identification number, bed position (imaging range), and low/high voltage”, and stored in memory circuitry 9, as needed.

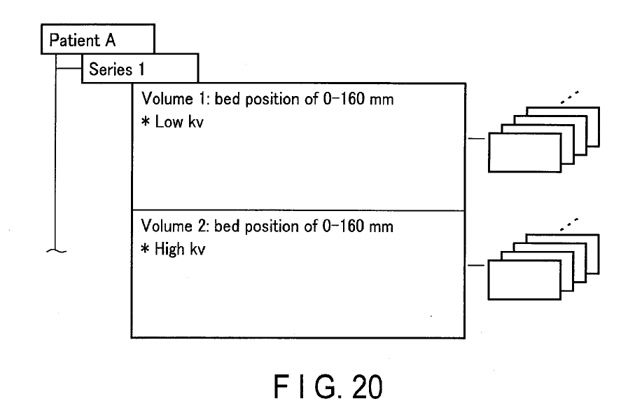

FIG. 20 is a schematic view showing the data structure of image data acquired by the image acquisition processing in steps Se1 to Se3 and stored in the memory circuitry 9. In the example shown in FIG. 20, two volumes obtained at the same bed position (same imaging range) under different imaging conditions (tube voltages) are stored in series 1.

Referring to FIG. 20, volumes 1, 2, . . . are labeled. For example, volume 1 belonging to series 1 is added with additional information including “bed position of 0-160 mm and low voltage (Low kV)”. Volume 2 belonging to series 2 is added with additional information including “bed position of 0-160 mm and high voltage (High kV)”.

As shown in FIG. 19, in step Se4, the user inputs determination conditions necessary to set transfer priority levels (transfer ordinal numbers) via input IF circuitry 11. For example, the user inputs, as determination conditions, “same bed position (same imaging range), dual energy imaging (imaging at different voltages), and same slice” via the input IF circuitry 11. Alternatively, the user may select preset conditions as “priority transfer (frame unit) for dual energy image analysis processing”. In step Se5, the user presses a processing start button via the input IF circuitry 11. In step Se6, using pressing of the processing start button as a trigger, the input determination conditions are confirmed. In step Se7, using pressing of the processing start button as a trigger, the input IF circuitry 11 outputs a processing start instruction to processing circuitry 15.

After pressing of the processing start button, the processing circuitry 15 collates, in step Se8, the confirmed determination conditions with the additional information associated with each of the plurality of volumes. Based on the collation result, the processing circuitry 15 sets a transfer priority level for each of the plurality of frames belonging to series 1 acquired in the study.

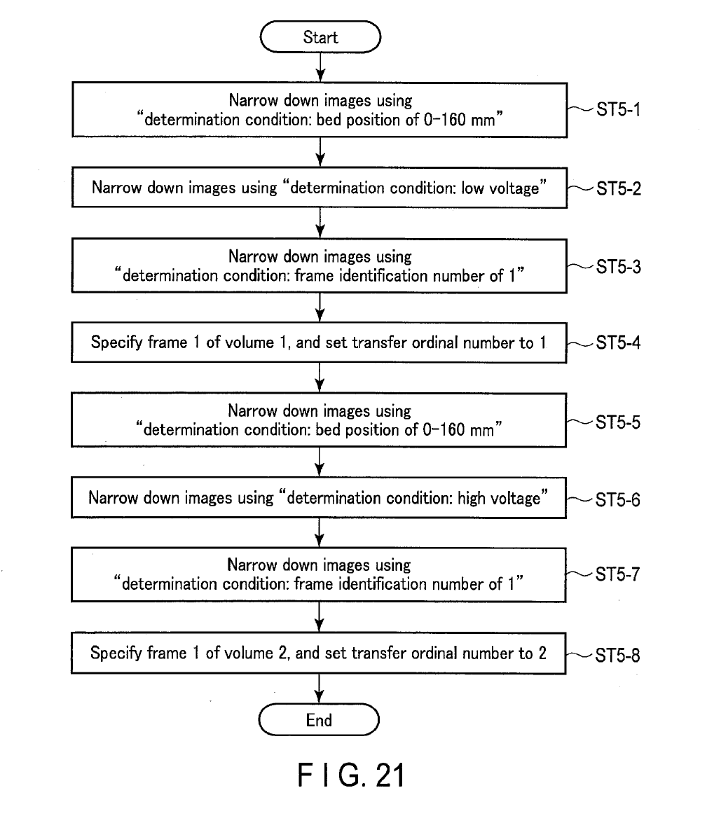

That is, as shown in FIG. 21, if the conditions “same bed position (same imaging range), dual energy imaging (imaging at different voltages), and same slice” are confirmed as determination conditions, the processing circuitry 15 narrows down whether there are volumes corresponding to pieces of additional information each satisfying the determination conditions. More specifically, in step ST5-1, medical images are narrowed down to those associated with “additional information: bed position of 0-160 mm” using “determination condition: bed position of 0-160 mm”. Furthermore, in step ST5-2, the medical images are narrowed down to those associated with “additional information: low voltage” using “determination condition: low voltage”. Furthermore, in step ST5-3, the medical images are narrowed down to those associated with “additional information: frame 1” using “determination condition: frame identification number of 1”. With these operations, in step ST5-4, frame 1 of volume 1 belonging to series 1 is specified as a transfer target satisfying the determination conditions, and the transfer ordinal number of frame 1 of volume 1 is set to 1.

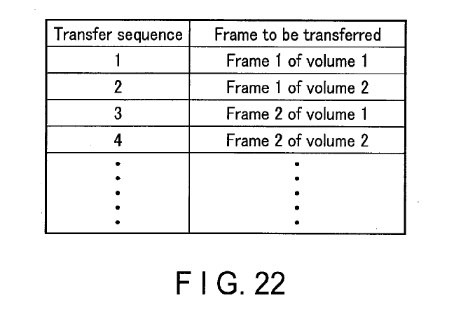

In step ST5-5, the medical images are narrowed down to those associated with “additional information: bed position of 0-160 mm” using “determination condition: bed position of 0-160 mm”. Furthermore, in step ST5-6, the medical images are narrowed down to those associated with “additional information: high voltage” using “determination condition: high voltage”. Furthermore, in step ST5-7, the medical images are narrowed down to those associated with “additional information: frame 1” using “determination condition: frame identification number of 1”. With these operations, in step ST5-8, frame 1 of volume 2 belonging to series 1 is specified as a transfer target satisfying the determination conditions, and the transfer ordinal number of frame 1 of volume 2 is set to 2. The same processing as that shown in FIG. 21 is repeatedly executed. That is, a transfer sequence is set so as to preferentially transfer the medical image data of the same slice at the same bed position, which allow comparison between an image captured at a low voltage and an image captured at a high voltage. This generates a transfer table, shown in FIG. 22, in which transfer ordinal numbers are respectively associated with frames to be transferred.

The transfer sequence shown in FIG. 22 is an example. The present invention is not limited to this. For example, the transfer sequence may be changed, as needed, to “frame 1 of volume 2→frame 1 of volume 1 . . . ”, “frame 2 of volume 1→frame 2 of volume 2 . . . ”, or the like.

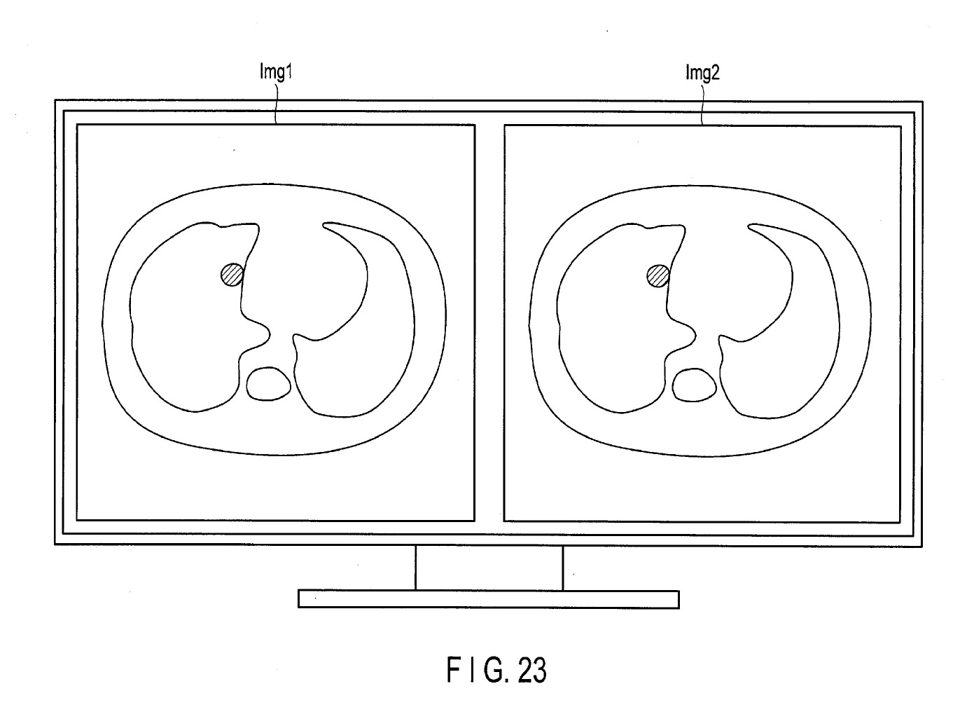

FIG. 23 is a view showing an example of a display mode of dual energy images transferred from the medical image diagnosis apparatus 1 to a WS 500. For example, as shown in FIG. 23, an image Img1 captured at a low voltage and an image Img2 captured at a high voltage are displayed side by side on the display circuitry of the WS 500.

With the above-described arrangement, the following effects can be obtained.

The medical image diagnosis apparatus 1 according to Example 5 includes the memory circuitry 9, the processing circuitry 15, and the transfer circuitry 17. The memory circuitry 9 stores volume 1 belonging to series 1 (low voltage) and obtained by imaging using dual energy, volume 2 belonging to series 2 (high voltage) and obtained by imaging using dual energy, and a plurality of pieces of additional information respectively associated with the volumes. The processing circuitry 15 collates each of the pieces of additional information with the predetermined determination conditions input via the input IF circuitry 11, determines a transfer priority level for each of the plurality of frames included in each volume, and sets a transfer ordinal number for each frame based on the determined priority level. For example, the processing circuitry 15 sets a transfer sequence so as to preferentially transfer frames to be compared by the WS 500 (that is, so as to preferentially transfer frames of the same slice at the same bed position in series 1). The transfer circuitry 17 transfers the plurality of frames to the WS 500 in the transfer sequence. This makes it possible to preferentially transfer frames necessary for image comparison. As a result, since the medical image diagnosis apparatus 1 according to Example 5 performs transfer processing for each frame in the same series, it can shorten the time until image comparison and display. Even if image transfer is interrupted midway, processing (display) can be executed using medical images received so far.

Note that the system control circuitry 21 uses the bed position as additional information for performing imaging according to the predetermined imaging sequence and setting the priority levels. The present invention, however, is not limited to this. The system control circuitry 21 may perform imaging and setting operations using, for example, patient coordinates obtained by setting a predetermined position of the patient as an origin.

### Example 6

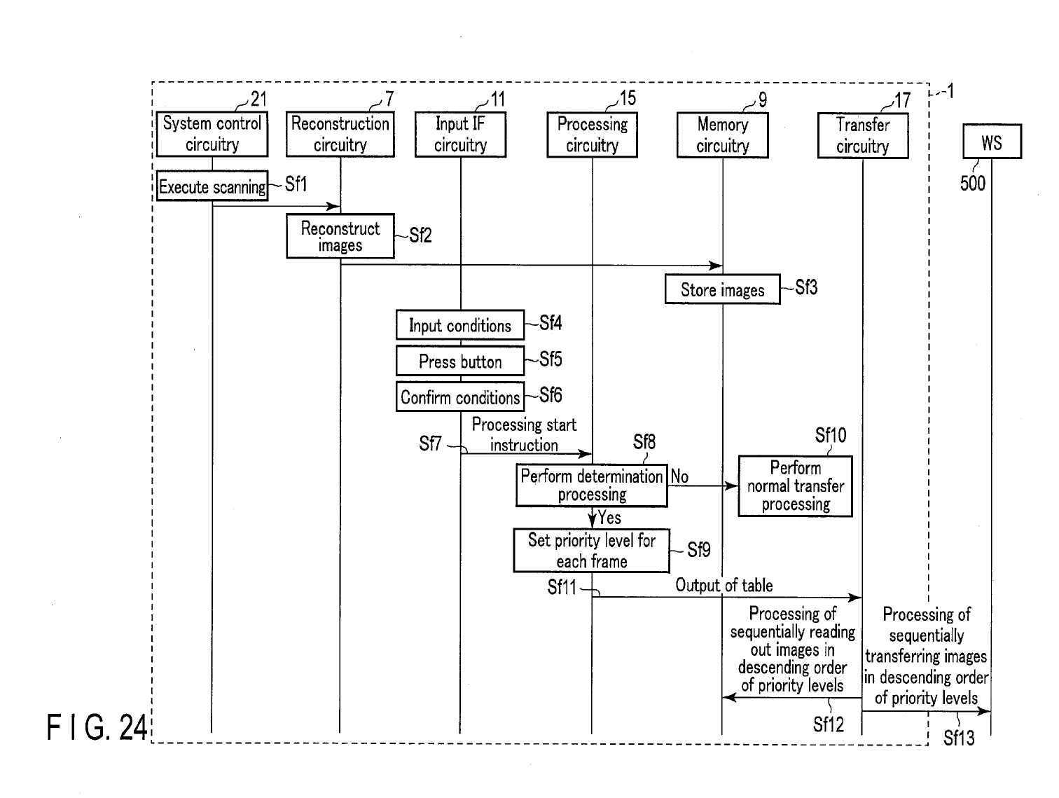

FIG. 24 is a sequence chart showing a procedure until a medical image diagnosis apparatus 1 executes predetermined studies (for example, examinations of different studies, such as examinations before/after surgery), and transfers data across the plurality of series obtained in the studies according to Example 6. Transfer processing executed by the medical image diagnosis apparatus 1 will be described below with reference to FIG. 24. The medical image diagnosis apparatus 1 according to Example 6 can set a transfer ordinal number for each frame included in volumes across the plurality of series. As an example, a description will be given by assuming priority transfer (frame unit) for analysis processing of images before/after surgery. Note that points common to the above examples will not be described in detail, and will be described, as needed.

As shown in FIG. 24, in step Sf1, after patient conditions and predetermined imaging conditions (a diagnosis portion, an imaging method, a tube voltage, a tube current, reconstruction parameters, contrast absence/presence, an imaging range (bed position), a cardiac phase, and the like) are input, system control circuitry 21 executes imaging within a predetermined range (for example, a bed position of 0 to 160 mm) in accordance with a predetermined imaging sequence. As a result, for example, volume 1 (as raw data) belonging to series 1 about examination 1 (study 1) and volume 2 (as row data) belonging to series 1 about examination 2 (study 2) are acquired. Note that each volume is formed from a plurality of two-dimensional data in frame units.

In step Sf2, reconstruction circuitry 7 executes image reconstruction processing using the respective acquired volumes and the set reconstruction parameters, and generates volume 1 belonging to series 1 about examination 1 (study 1) and volume 2 belonging to series 1 about examination 2 (study 2). In step Sf3, each generated volume of each series is added with additional information including “examination ID, volume identification number, frame identification number, bed position (imaging range), and before/after surgery”, and stored in memory circuitry 9, as needed.

Note that if there is a period until imaging after surgery, each volume with the additional information including “examination ID, volume identification number, frame identification number, bed position (imaging range), and before surgery”, which has been stored in step Sf3, may be stored in an externally provided storage device instead of the memory circuitry 9. The volume stored in the externally provided storage device may be read out every time image transfer is executed.

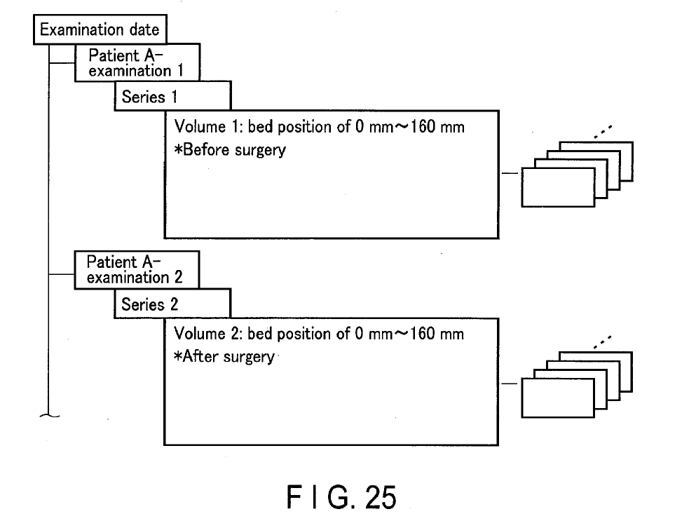

FIG. 25 is a schematic view showing the data structure of image data acquired by the image acquisition processing in steps Sf1 to Sf3 and stored in the memory circuitry 9. In the example shown in FIG. 25, volume 1 acquired before surgery is stored in series 1. Volume 2 acquired after surgery is stored in series 2.

Referring to FIG. 25, volumes 1, 2, . . . are labeled. For example, volume 1 belonging to series 1 about examination 1 is added with, for example, additional information including “examination number of 1, bed position of 0-160 mm, and before surgery”. Volume 2 belonging to series 2 about examination 2 is added with additional information including “examination number of 2, bed position of 0-160 mm, and after surgery”.

As shown in FIG. 24, in step Sf4, the user inputs determination conditions necessary to set transfer priority levels (transfer ordinal numbers) via input IF circuitry 11. For example, the user inputs, as determination conditions, “different examinations (before/after surgery), same bed position, and same slice” via the input IF circuitry 11. Alternatively, the user may select preset conditions as “priority transfer (frame unit) for analysis processing of images before/after surgery”. In step Sf5, the user presses a processing start button via the input IF circuitry 11. In step Sf6, using pressing of the processing start button as a trigger, the input determination conditions are confirmed. In step Sf7, using pressing of the processing start button as a trigger, the input IF circuitry 11 outputs a processing start instruction to processing circuitry 15.

After pressing of the processing start button, the processing circuitry 15 collates, in step Sf8, the confirmed determination conditions with the additional information associated with each of the plurality of volumes. Based on the collation result, the processing circuitry 15 sets a transfer priority level for each volume belonging to series 1 acquired in the plurality of studies.

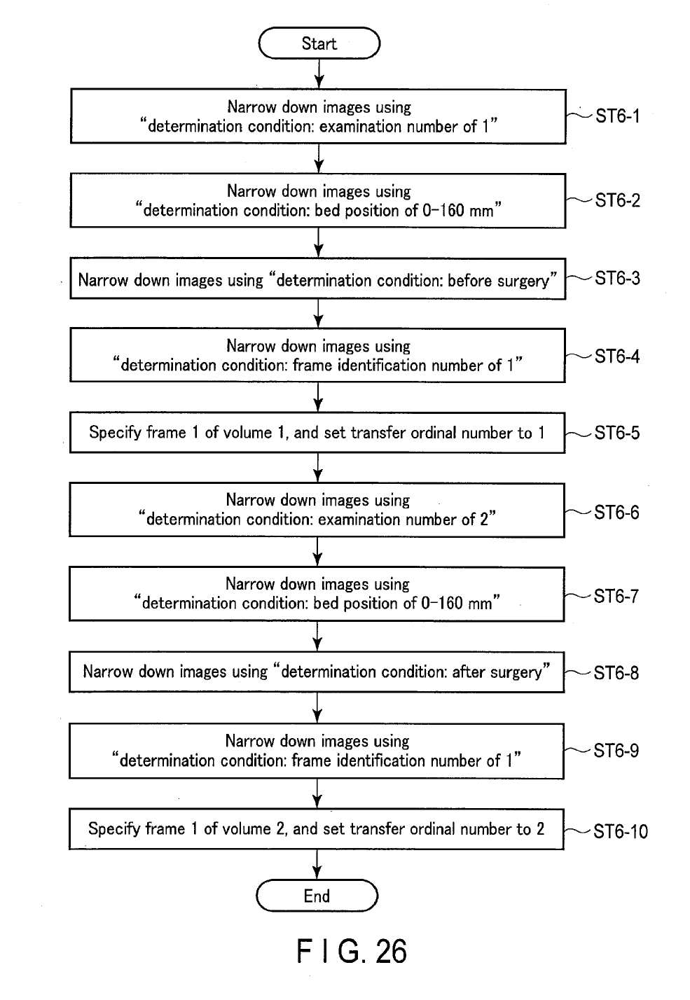

That is, as shown in FIG. 26, if the conditions “different examinations (before/after surgery), same bed position, and same slice” are confirmed as determination conditions, the processing circuitry 15 narrows down whether there are volumes corresponding to pieces of additional information each satisfying the determination conditions. More specifically, in step ST6-1, medical images are narrowed down to those associated with “additional information: examination 1” using “determination condition: examination number of 1”. In step ST6-2, the medical images are narrowed down to those associated with “additional information: bed position of 0-160 mm” using “determination condition: bed position of 0-160 mm”. Furthermore, in step ST6-3, the medical images are narrowed down to those associated with “additional information: before surgery” using “determination condition: before surgery”. In step ST6-4, the medical images are narrowed down to those associated with “additional information: frame 1” using “determination condition: frame identification number of 1”. With these operations, in step ST6-5, frame 1 of volume 1 belonging to series 1 about examination 1 is specified as a transfer target satisfying the determination conditions, and the transfer ordinal number of frame 1 of volume 1 is set to 1.

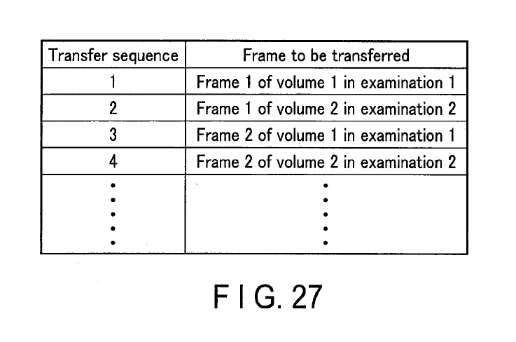

In step ST6-6, the medical images are narrowed down to those associated with “additional information: examination 2” using “determination condition: examination 2”. Furthermore, in step ST6-7, the medical images are narrowed down to those associated with “additional information: bed position of 0-160 mm” using “determination condition: bed position of 0-160 mm”. Furthermore, in step ST6-8, the medical images are narrowed down to those associated with “additional information: after surgery” using “determination condition: after surgery”. In step ST6-9, the medical images are narrowed down to those associated with “additional information: frame 1” using “determination condition: frame identification number of 1”. With these operations, in step ST6-10, frame 1 of volume 2 belonging to series 2 about examination 2 is specified as a transfer target satisfying the determination conditions, and the transfer ordinal number of frame 1 of volume 2 is set to 2. The same processing as that shown in FIG. 26 is repeatedly executed. That is, a transfer sequence is set so as to preferentially transfer the medical image data of the same slice at the same bed position, which allows comparison between images before and after surgery. This generates a transfer table, shown in FIG. 27, in which transfer ordinal numbers are respectively associated with frames to be transferred.

The transfer sequence shown in FIG. 27 is an example. The present invention is not limited to this. For example, the transfer sequence may be changed, as needed, to “frame 1 of volume 2 in examination 2→frame 1 of volume 1 in examination 1 . . . ”, “frame 2 of volume 1 in examination 1→frame 2 of volume 2 in examination 2 . . . ”, or the like.

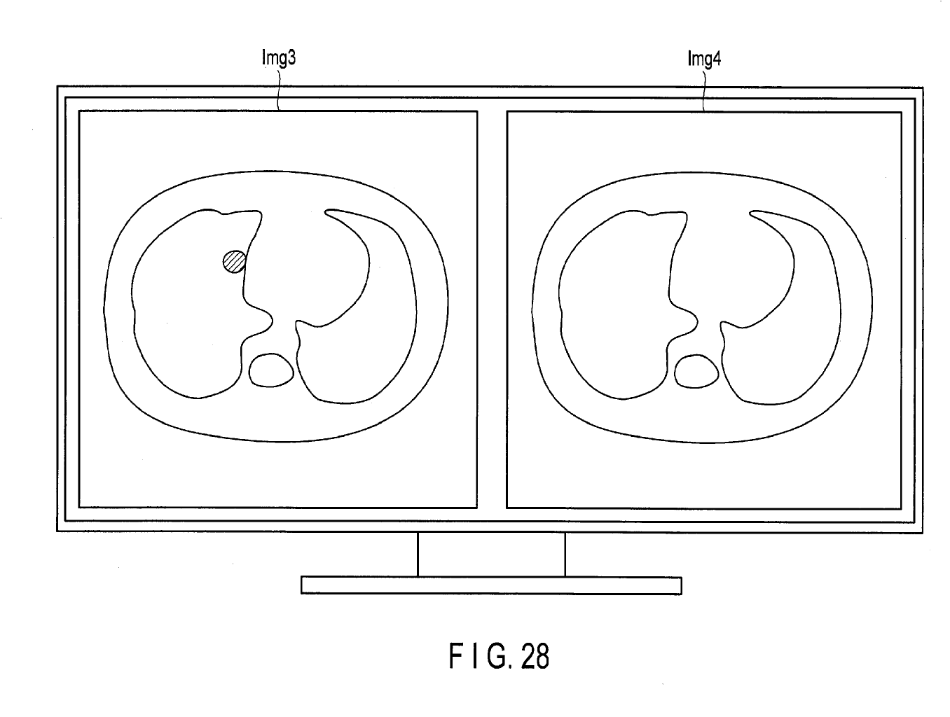

FIG. 28 is a view showing an example of a display mode of images before/after surgery transferred from the medical image diagnosis apparatus 1 to a WS 500. For example, as shown in FIG. 28, an image Img3 captured before surgery and an image Img4 captured after surgery are displayed side by side on the display circuitry of the WS 500.

With the above-described arrangement, the following effects can be obtained.

The medical image diagnosis apparatus 1 according to Example 6 includes the memory circuitry 9, the processing circuitry 15, and transfer circuitry 17. The memory circuitry 9 stores volume 1 belonging to series 1 (before surgery) about examination 1, volume 2 belonging to series 2 (after surgery) about examination 1, and a plurality of pieces of additional information respectively associated with the volumes. The processing circuitry 15 collates each of the pieces of additional information with the predetermined determination conditions input via the input IF circuitry 11, determines a transfer priority level for each of the plurality of frames included in each volume, and sets a transfer ordinal number of each frame based on the determined priority level. For example, the processing circuitry 15 sets a transfer sequence so as to preferentially transfer frames to be compared by the WS 500 (that is, so as to preferentially transfer frames of the same slice at the same bed position in series 1 of each study). The transfer circuitry 17 transfers the plurality of frames to the WS 500 in the transfer sequence. This makes it possible to preferentially transfer frames necessary for image comparison. As a result, since the medical image diagnosis apparatus 1 according to Example 6 transfers each frame across the different studies, it can shorten the time until image comparison and display. Even if image transfer is interrupted midway, processing (display) can be executed using medical images received so far.

Note that the system control circuitry 21 uses the bed position as additional information for performing imaging according to the predetermined imaging sequence and setting the priority levels. The present invention, however, is not limited to this. The system control circuitry 21 may perform imaging and setting operations using, for example, patient coordinates obtained by setting a predetermined position of the patient as an origin.

### Example 7

In Examples 1 to 6 described above, using pressing of a processing start button as a trigger, setting processing starts. The present invention, however, is not limited to this. A medical image diagnosis apparatus 1 according to Example 7 can set transfer priority levels without using pressing of a processing start button as a trigger, thereby transferring images.

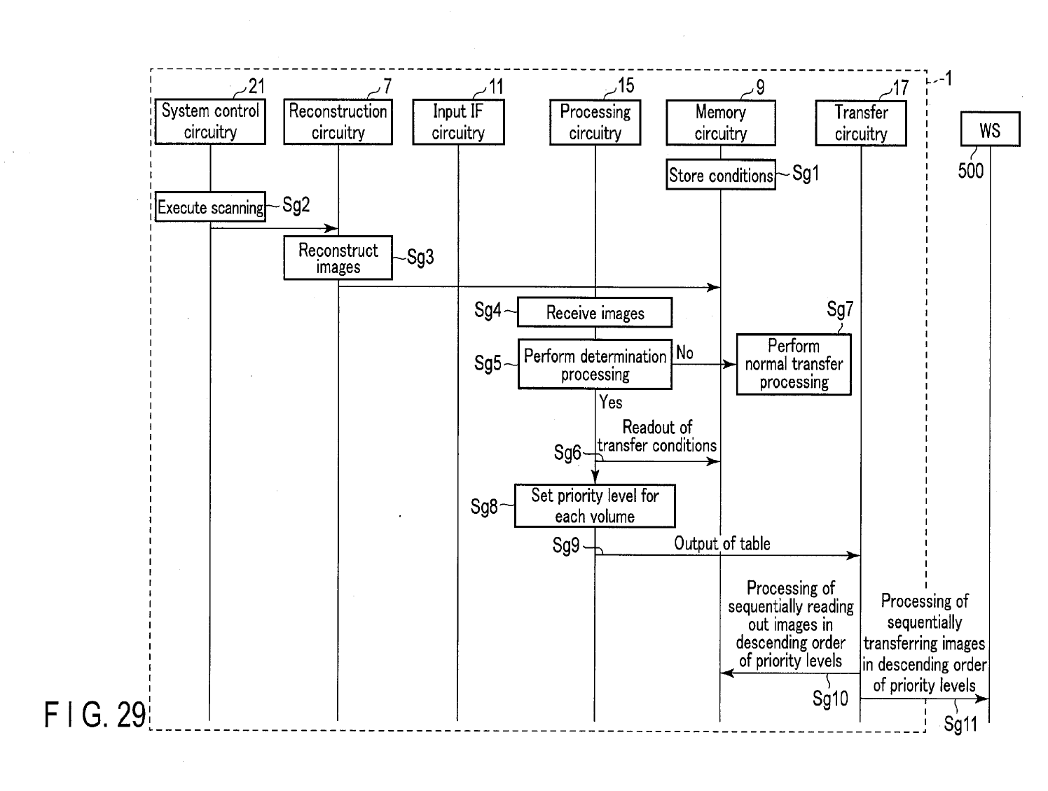

FIG. 29 is a sequence chart showing a procedure until a medical image diagnosis apparatus 1 executes a predetermined study (for example, an examination of a circulatory system using a contrast medium), and transfers data across a plurality of series obtained in the study according to Example 7. Transfer processing executed by the medical image diagnosis apparatus 1 will be described below with reference to FIG. 29. Note that points common to the above examples will not be described in detail, and will be described, as needed.

As shown in FIG. 29, in step Sg1, memory circuitry 9 stores in advance determination conditions necessary to set transfer priority levels (transfer ordinal numbers).

As shown in FIG. 29, in step Sg2, after patient conditions and predetermined imaging conditions (a diagnosis portion, an imaging method, a tube voltage, a tube current, reconstruction parameters, contrast absence/presence, an imaging range (bed position), a cardiac phase, and the like) are input, system control circuitry 21 executes imaging in accordance with a predetermined imaging sequence. As a result, for example, a plurality of volumes (as raw data) are acquired. In step Sg3, the reconstruction circuitry 7 executes image reconstruction processing using the respective acquired volumes and the set reconstruction parameters, and generates a plurality of volumes. In step Sg4, each of the plurality of generated volumes is added with additional information, and output to processing circuitry 15. Furthermore, in step Sg5, using reception of the plurality of volumes as a trigger, the processing circuitry 15 determines whether the plurality of received volumes include volumes each satisfying the determination conditions. If it is determined that the plurality of received volumes include volumes each satisfying the determination conditions (YES in step Sg5), the processing circuitry 15 reads out the input determination conditions in step Sg6.

In step Sg8, the processing circuitry 15 collates the readout determination conditions with each of the pieces of additional information respectively associated with the plurality of volumes. Based on the collation result, the processing circuitry 15 sets a transfer priority level for each of the plurality of volumes belonging to each series acquired in the study.

With the above-described arrangement, the following effects can be obtained.

The medical image diagnosis apparatus 1 according to Example 7 includes the memory circuitry 9, the processing circuitry 15, and transfer circuitry 17. The memory circuitry 9 stores a plurality of medical images, a plurality of pieces of additional information respectively associated with the plurality of medical images, and the predetermined determination conditions. The processing circuitry 15 collates each of the plurality of pieces of additional information with the stored predetermined conditions, and determines a transfer priority level for each of the plurality of medical images, thereby setting a transfer ordinal number for each frame based on the determined priority level. The transfer circuitry 17 transfers, in accordance with the transfer ordinal numbers, the plurality of medical images to at least one of a WS 500 and a PACS 700 which are connected via a network NW. As a result, the medical image diagnosis apparatus 1 according to Example 7 can set transfer priority levels based on the stored determination conditions without any trigger for instructing start of processing, thereby transferring the images in descending order of the set priority levels.

Note that the system control circuitry 21 uses the bed position as additional information for performing imaging according to the predetermined imaging sequence and setting the priority levels. The present invention, however, is not limited to this. The system control circuitry 21 may perform imaging and setting operations using, for example, patient coordinates obtained by setting a predetermined position of the patient as an origin.

### Second Embodiment

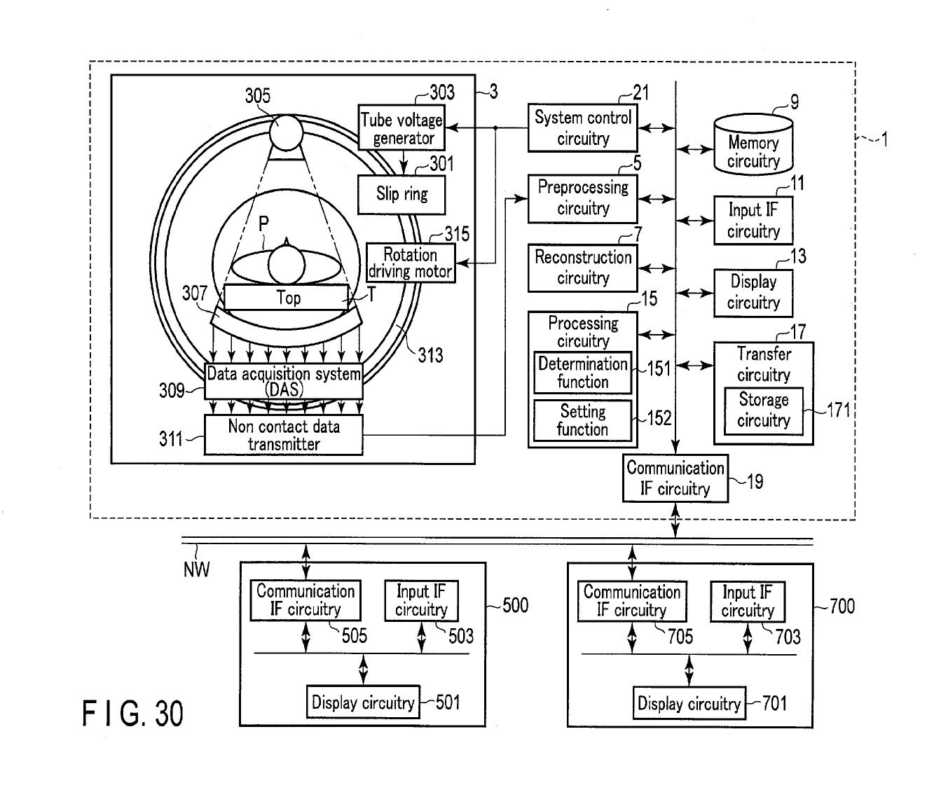

FIG. 30 is a block diagram showing a medical image transfer system according to the second embodiment. Note that parts common to the first embodiment will not be described in detail.

As shown in FIG. 30, the medical image transfer system includes a medical image diagnosis apparatus 1, a medical image analysis apparatus (workstation (WS)) 500 for executing image data analysis, and a PACS 700 for saving medical images. For example, the medical image diagnosis apparatus 1, WS 500, and PACS 700 are interconnected via a network NW. Note that FIG. 30 shows the arrangement of an X-ray CT apparatus as an example of the medical image diagnosis apparatus 1. Note also that the second embodiment shows the arrangement of the X-ray CT apparatus as an example of the medical image diagnosis apparatus 1 but the present invention is not limited to this. For example, the medical image diagnosis apparatus 1 may be an MRI apparatus, X-ray diagnosis apparatus, nuclear medicine diagnosis apparatus, or ultrasonic diagnosis apparatus.

The WS 500 includes display circuitry 501, input IF circuitry 503, and communication IF circuitry 505.

The display circuitry 501 displays, on a display device, a list (including additional information) about images stored in memory circuitry 9, a CT image, and the like. As the display device, a CRT, LCD, OELD, plasma display, or the like can be used, as needed.

The input IF circuitry 503 serves as an interface for inputting a command or the like corresponding to a user operation. For example, by inputting a command or the like corresponding to a user operation via the input IF circuitry 503 with reference to the list displayed on the display circuitry 501, information (determination conditions) necessary for the user to set transfer priority levels (transfer ordinal numbers) is input. The input IF circuitry 503 includes, for example, a keyboard, a mouse, a touch panel, a trackball, and various buttons.

The communication IF circuitry 505 communicates with an external apparatus by wired or wireless connection. The external apparatus is, for example, a modality, a server included in a system such as an RIS, HIS, or PACS, or another workstation. In the second embodiment, for example, the communication IF circuitry 505 communicates with the medical image diagnosis apparatus 1 and the PACS 700.

The PACS 700 includes display circuitry 701, input IF circuitry 703, and communication IF circuitry 705.

The display circuitry 701 displays, on a display device, a list (including additional information) about images stored in the memory circuitry 9, a CT image, and the like. As the display device, a CRT, LCD, OELD, plasma display, or the like can be used, as needed.

The input IF circuitry 703 serves as an interface for inputting a command or the like corresponding to a user operation. For example, by inputting a command or the like corresponding to a user operation via the input IF circuitry 703 with reference to the list displayed on the display circuitry 701, information (determination conditions) necessary for the user to set transfer priority levels (transfer ordinal numbers) is input. The input IF circuitry 503 includes, for example, a keyboard, a mouse, a touch panel, a trackball, and various buttons.

The communication IF circuitry 705 communicates with an external apparatus by wired or wireless connection. The external apparatus is, for example, a modality, a server included in a system such as an RIS, HIS, or PACS, or another workstation. In the second embodiment, for example, the communication IF circuitry 705 communicates with the medical image diagnosis apparatus 1 and the WS 500.

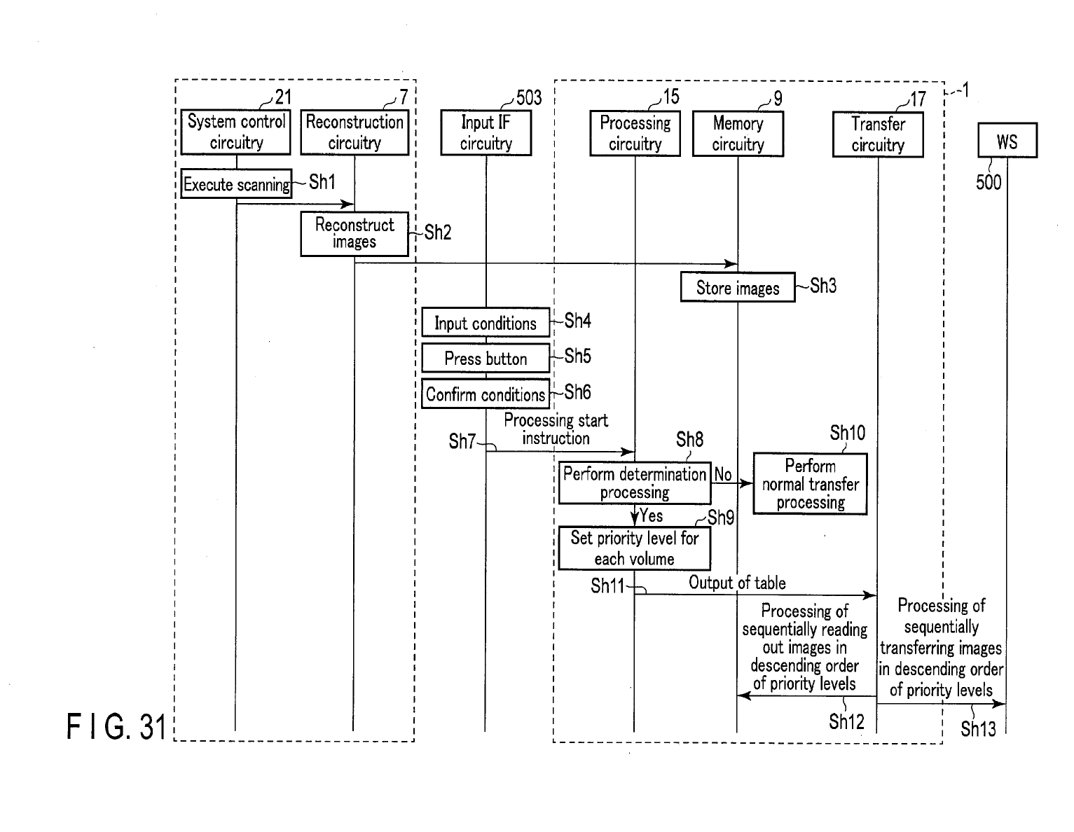

FIG. 31 is a sequence chart showing a procedure until the medical image diagnosis apparatus 1 executes a predetermined study (for example, a cardiac examination using a contrast medium), and transfers data across a plurality of series obtained in the study according to the second embodiment. Transfer processing executed by the medical image diagnosis apparatus 1 will be described below with reference to FIG. 31.

As shown in FIG. 31, in step Sh1, after patient conditions and predetermined imaging conditions (a diagnosis portion, an imaging method, a tube voltage, a tube current, reconstruction parameters, contrast absence/presence, an imaging range (bed position), cardiac phase, and the like) are input, system control circuitry 21 executes pre-contrast imaging and post-contrast imaging within a predetermined range (for example, a bed position of 0 to 300 mm) in accordance with a predetermined imaging sequence. As a result, for example, volumes 1, 2, and 3 (as raw data) belonging to series 1 are acquired in pre-contrast imaging, and volumes 4, 5, and 6 (as raw data) belonging to series 2 are acquired in post-contrast imaging. In step Sh2, the reconstruction circuitry 7 executes image reconstruction processing using the respective acquired volumes and the set reconstruction parameters, and generates volumes 1, 2, and 3 belonging to series 1 by non-contrast imaging and volumes 4, 5, and 6 belonging to series 2 by contrast imaging. In step Sh3, each generated volume of each series is added with additional information including “volume identification number, contrast absence/presence, bed position (imaging range), and cardiac phase in ECG waveform”, and stored in the memory circuitry 9, as needed.

As shown in FIG. 31, in step Sh4, the user inputs determination conditions necessary to set transfer priority levels (transfer ordinal numbers) via the input IF circuitry 503. In step Sh5, the user presses a processing start button via the input IF circuitry 503. In step Sh6, using pressing of the processing start button as a trigger, the input determination conditions are confirmed. In step Sh7, using pressing of the processing start button as a trigger, the input IF circuitry 503 outputs a processing start instruction to processing circuitry 15.

After pressing of the processing start button, the processing circuitry 15 collates, in step Sh8, the confirmed determination conditions with the additional information associated with each of the plurality of volumes. In step Sh9, based on the collation result, the processing circuitry 15 sets a transfer priority level for each of the plurality of volumes belonging to each series acquired in the study.

In step Sh11, the processing circuitry 15 outputs a transfer table to transfer circuitry 17. In step Sh12, the transfer circuitry 17 sequentially reads out, from the memory circuitry 9, the volumes having higher transfer priority levels in accordance with the received transfer table. In response to the image readout processing from the transfer circuitry 17, the memory circuitry 9 outputs a plurality of corresponding volumes to the transfer circuitry 17. In step Sh13, the transfer circuitry 17 transfers, to the WS 500 as a request source, the volumes read out in accordance with the transfer table.

With the above-described arrangement, the following effects can be obtained.

The medical image diagnosis apparatus 1 according to the second embodiment includes the memory circuitry 9, processing circuitry 15, and transfer circuitry 17. The memory circuitry 9 stores a plurality of medical images, and a plurality of pieces of additional information respectively associated with the plurality of medical images. The processing circuitry 15 collates each of the plurality of pieces of additional information with the predetermined determination conditions input to the input IF circuitry 503, and determines a transfer priority level for each of the plurality of medical images, thereby setting a transfer ordinal number of each frame based on the determined priority level. The transfer circuitry 17 transfers, in accordance with the transfer ordinal numbers, the plurality of medical images to the WS 500 via the network NW. As a result, even if image transfer is interrupted midway, the medical image diagnosis apparatus 1 according to the second embodiment can execute processing (display) using medical images received so far.

Note that the system control circuitry 21 uses the bed position as additional information for performing imaging according to the predetermined imaging sequence and setting the priority levels. The present invention, however, is not limited to this. The system control circuitry 21 may perform imaging and setting operations using, for example, patient coordinates obtained by setting a predetermined position of the patient as an origin.

In the above embodiment, the medical image diagnosis apparatus 1 is a so-called third generation. That is, the medical image diagnosis apparatus 1 is a rotate/rotate-type apparatus in which an X-ray tube 305 and an X-ray detector 307 integrally rotate around a rotation axis. However, the medical image diagnosis apparatus 1 according to this embodiment is not limited to this. For example, the medical image diagnosis apparatus 1 may be a stationary/rotate-type apparatus in which a number of light-receiving bands arrayed in a ring shape are fixed and only the X-ray tube 305 rotates around the rotation axis. Alternatively, the medical image diagnosis apparatus 1 may be a fifth generation in which a number of light-receiving bands arrayed in a ring shape are fixed, and anodes are arranged in a ring shape, and irradiated with electron beams by electromagnetic deflection.

Furthermore, the term “predetermined processor” used in the above description indicates, for example, a dedicated or general-purpose processor, circuit (circuitry), processing circuit (circuitry), operation circuit (circuitry), arithmetic circuit (circuitry), ASIC (Application Specific Integrated Circuit), programmable logic device (for example, SPLD: Simple Programmable Logic Device), CPLD (Complex Programmable Logic Device), or FPGA (Field Programmable Gate Array), or the like. Each component (each processing circuitry) of this embodiment may be implemented by a plurality of processors without limitation to a single processor. Furthermore, a plurality of components (a plurality of processing circuitry) may be implemented by a single processor.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A medical image processing apparatus, comprising:
memory circuitry configured to store a plurality of medical images obtained by capturing a subject and a plurality of pieces of additional information respectively associated with the plurality of medical images; and
transfer circuitry configured to transfer the plurality of medical images to an external apparatus in accordance with a predetermined transfer sequence,
wherein if the additional information associated with each medical image matches a predetermined determination condition, the transfer circuitry changes the predetermined transfer sequence, and transfers the plurality of medical images to the external apparatus.

2. The apparatus of claim 1, wherein
the transfer circuitry includes storage circuitry configured to store transfer requests about the plurality of medical images to the external apparatus,
if each of pieces of additional information respectively associated with medical images corresponding to the transfer requests stored in the storage circuitry matches the predetermined determination condition, the transfer circuitry changes the transfer sequence corresponding to a reception sequence of the transfer requests stored in the storage circuitry and transfers the plurality of medical images, and
if each of the pieces of additional information respectively associated with the medical images corresponding to the transfer requests stored in the storage circuitry does not match the predetermined determination condition, the transfer circuitry transfers the plurality of images using, as the predetermined transfer sequence, a transfer sequence corresponding to the reception sequence of the transfer requests stored in the storage circuitry.

3. The apparatus of claim 2, wherein the transfer circuitry sets the predetermined transfer sequence based on at least one of an examination list display sequence and a storage sequence of the medical images to be transferred, which are stored in the storage circuitry.

4. The apparatus of claim 1, further comprising:
processing circuitry configured to collate each of the pieces of additional information with the predetermined determination condition, and sets a transfer sequence with respect to the plurality of medical images associated with the pieces of additional information each matching the predetermined determination condition.

5. The apparatus of claim 4, wherein the processing circuitry
collates each of the pieces of additional information with the predetermined determination condition to determine transfer priority levels about the plurality of medical images associated with the pieces of additional information each matching the predetermined determination condition, and
sets a transfer sequence about the plurality of medical images by the transfer circuitry in accordance with the determined priority levels.

6. The apparatus of claim 4, wherein the memory circuitry stores, as the plurality of medical images, a plurality of volume images each including a plurality of frame images, and stores the additional information in association with each of the plurality of volume images.

7. The apparatus of claim 6, wherein the processing circuitry sets the transfer sequence so as to alternately transfer the plurality of frame images for each volume image between different volume images of different series among the plurality of volume images respectively associated with the pieces of matching additional information.

8. The apparatus of claim 7, wherein the processing circuitry sets the transfer sequence so as to alternately transfer at least one frame image from the different volume images.

9. The apparatus of claim 4, wherein the storage circuitry stores, as the plurality of medical images, a plurality of volume images each including a plurality of frame images, and stores the additional information in association with each of the plurality of frame images.

10. The apparatus of claim 9, wherein the processing circuitry sets the transfer sequence so as to alternately transfer the plurality of frame images for each frame image between different volume images of different series among the plurality of volume images respectively associated with the pieces of matching additional information.

11. The apparatus of claim 10, wherein the processing circuitry sets the transfer sequence so as to alternately transfer at least one frame image from the different volume images.

12. The apparatus of claim 9, wherein the processing circuitry sets the transfer sequence so as to alternately transfer the plurality of frame images for each frame image between different volume images of the same series among the plurality of volume images respectively associated with the pieces of matching additional information.

13. The apparatus of claim 12, wherein the processing circuitry sets the transfer sequence so as to alternately transfer at least one frame image from the different volume images.

14. The apparatus of claim 4, wherein the memory circuitry stores a plurality of frame images as the plurality of medical images, and stores the additional information in association with each of the plurality of frame images.

15. The apparatus of claim 14, wherein the processing circuitry sets the transfer sequence so as to alternately transfer the plurality of frame images between different series among the plurality of frame images respectively associated with the pieces of matching additional information.

16. A medical image transfer system comprising a medical image processing apparatus and an external apparatus which are connected via a network,
the medical image processing apparatus comprising
memory circuitry configured to store a plurality of medical images obtained by capturing a subject and a plurality of pieces of additional information respectively associated with the plurality of medical images, and
transfer circuitry configured to transfer the plurality of medical images to an external apparatus in accordance with a predetermined transfer sequence,
wherein if the additional information associated with each medical image matches a predetermined determination condition, the transfer circuitry changes the predetermined transfer sequence, and transfers the plurality of medical images to the external apparatus, and
the external apparatus comprising
input circuitry configured to receive the predetermined determination condition from a user, and
reception circuitry configured to receive, from the transfer circuitry, the plurality of medical images each satisfying the predetermined determination condition input to the input circuitry.
